SEQUENCE LISTING (28 sequences)

SEQ ID NO: 1 - DNA, 428 bp, Artificial Sequence
  Description of Artificial Sequence Synthetic polynucleotide
  CDS (1)..(426)
atgagggtccagattcagtttctggggctccttctgctctggacatcagttgtccagtgtgatgtccagatgacccagtctccatcttatcttgctacgtctcctggagagagtgtttccatcagttgcaaggcaagtaaaaacattgacacatacttagcctggtatcaggagaaacctgggaaaacgaataagcttcttatctactctgggtcaactttgcaatctggaactccatcgagattcagtggcagtggatctggtacagatttcacgctcaccatcagaaacctggagtctgaagattttgcagtctactactgtcaacagcataatgaatacccgctcacgttcggttctgggaccaagctggagatcaaacgggctgatgctgcaccaactgtatccatcttcccaccatcctcgga

SEQ ID NO: 2 - protein, 142 aa, Artificial Sequence
  Description of Artificial Sequence Synthetic polypeptide
MRVQIQFLGLLLLWTSVVQCDVQMTQSPSYLATSPGESVSISCKASKNIDTYLAWYQEKPGKTNKLLIYSGSTLQSGTPSRFSGSGSGTDFTLTIRNLESEDFAVYYCQQHNEYPLTFGSGTKLEIKRADAAPTVSIFPPSS

SEQ ID NO: 3 - protein, 11 aa, Artificial Sequence
  Description of Artificial Sequence Synthetic peptide
KASKNIDTYLA

SEQ ID NO: 4 - protein, 5 aa, Artificial Sequence
  Description of Artificial Sequence Synthetic peptide
SGSTL

SEQ ID NO: 5 - protein, 9 aa, Artificial Sequence
  Description of Artificial Sequence Synthetic peptide
QQHNEYPLT

SEQ ID NO: 6 - DNA, 483 bp, Artificial Sequence
  Description of Artificial Sequence Synthetic polynucleotide
  CDS (1)..(483)
atggacaccaggctctgcttggttttccttgtccttttcataaaaggtgtccagtgtgaggtgcagctggtggagtctggtggaggcttagtgcagcctggaaggtccctgaaactctcctgtgcagcctcaggattcactttcagtaactattacatggcctgggtccgccaggctccaacgaagggtctggagtgggtcgcatccattagttttgagggtaatagaaatcactatggagactccgtgaagggccgaatcactatctccagagataatgcaaaaagcaccctatacctgcaaatgaccagtctgaggcctgaggacacggccacttattattgtgcaagacatcgggggtatagtacgaatttttatcacgacgttttggatgcctggggtcaaggagctttagtcactgtctcctcagctgaaacaacagccccatctgtctatccactggctcctggaactgctctcaaa

SEQ ID NO: 7 - protein, 161 aa, Artificial Sequence
  Description of Artificial Sequence Synthetic polypeptide
MDTRLCLVFLVLFIKGVQCEVQLVESGGGLVQPGRSLKLSCAASGFTFSNYYMAWVRQAPTKGLEWVASISFEGNRNHYGDSVKGRITISRDNAKSTLYLQMTSLRPEDTATYYCARHRGYSTNFYHDVLDAWGQGALVTVSSAETTAPSVYPLAPGTALK

SEQ ID NO: 8 - protein, 10 aa, Artificial Sequence
  Description of Artificial Sequence Synthetic peptide
GFTFSNYYMA

SEQ ID NO: 9 - protein, 16 aa, Artificial Sequence
  Description of Artificial Sequence Synthetic peptide
SISFEGNRNHYGDSVK

SEQ ID NO: 10 - protein, 19 aa, Artificial Sequence
  Description of Artificial Sequence Synthetic peptide
HRGYSTNFYHDVLDAWGQG

SEQ ID NO: 11 - protein, 646 aa, Homo sapiens
MGLPRLVCAFLLAACCCCPRVAGVPGEAEQPAPELVEVEVGSTALLKCGLSQSQGNLSHVDWFSVHKEKRTLIFRVRQGQGQSEPGEYEQRLSLQDRGATLALTQVTPQDERIFLCQGKRPRSQEYRIQLRVYKAPEEPNIQVNPLGIPVNSKEPEEVATCVGRNGYPIPQVIWYKNGRPLKEEKNRVHIQSSQTVESSGLYTLQSILKAQLVKEDKDAQFYCELNYRLPSGNHMKESREVTVPVFYPTEKVWLEVEPVGMLKEGDRVEIRCLADGNPPPHFSISKQNPSTREAEEETTNDNGVLVLEPARKEHSGRYECQAWNLDTMISLLSEPQELLVNYVSDVRVSPAAPERQEGSSLTLTCEAESSQDLEFQWLREETDQVLERGPVLQLHDLKREAGGGYRCVASVPSIPGLNRTQLVKLAIFGPPWMAFKERKVWVKENMVLNLSCEASGHPRPTISWNVNGTASEQDQDPQRVLSTLNVLVTPELLETGVECTASNDLGKNTSILFLELVNLTTLTPDSNTTTGLSTSTASPHTRANSTSTERKLPEPESRGVVIVAVIVCILVLAVLGAVLYFLYKKGKLPCRRSGKQEITLPPSRKTELVVEVKSDKLPEEMGLLQGSSGDKRAPGDQGEKYIDLRH

SEQ ID NO: 12 - DNA, 474 bp, Artificial Sequence
  Description of Artificial Sequence Synthetic polynucleotide
  CDS (1)..(474)
atggaatcacagacccaggtcctcatgtccctgctgctctggatttctggtacctgtggggacattgtgatgacccagtctccatcctctctggctgtgtcagctggggagacggtctctatacactgcaagtccagtcagagtcttttatacagtggaacccaaaagaactacttggcctggttccagcagaaaccaggacagtctcctaaactgctgatcttctgggcatctactaggcagtctggtgtccctgatcgcttcataggccgtggatctgggacagacttcactctgaccatcagcggtgtgcaggcagaagatctggcaatttattactgtcaacaatattatgatactctcacggacacgtttggagcggggaccaagctggaactgaaacgggctgatgctgcaccaactgtatctatcttcccaccatccacggaacagttagcaactggaggtgcctca

SEQ ID NO: 13 - protein, 158 aa, Artificial Sequence
  Description of Artificial Sequence Synthetic polypeptide
MESQTQVLMSLLLWISGTCGDIVMTQSPSSLAVSAGETVSIHCKSSQSLLYSGTQKNYLAWFQQKPGQSPKLLIFWASTRQSGVPDRFIGRGSGTDFTLTISGVQAEDLAIYYCQQYYDTLTDTFGAGTKLELKRADAAPTVSIFPPSTEQLATGGAS

SEQ ID NO: 14 - protein, 17 aa, Artificial Sequence
  Description of Artificial Sequence Synthetic peptide
KSSQSLLYSGTQKNYLA

SEQ ID NO: 15 - protein, 7 aa, Artificial Sequence
  Description of Artificial Sequence Synthetic peptide
WASTRQS

SEQ ID NO: 16 - protein, 10 aa, Artificial Sequence
  Description of Artificial Sequence Synthetic peptide
QQYYDTLTDT

SEQ ID NO: 17 - DNA, 469 bp, Artificial Sequence
  Description of Artificial Sequence Synthetic polynucleotide
  CDS (1)..(468)
atggacatcaggctcagcttggctttcctggtccttttcataaaaggtgtccagtgtgaggtgcggctggtggagtctgggggaggcttagtgcagcctggaaagtccatgaaactctcctgtgtagcctcgggattcaaattcagtaactattacatgtcctgggtccgccaggctccagcgaagggtctggagtgggtcgcatccattagtgatggtggtggtgacactttctgtcgagacttggtgaagggccgattcactatctccagagataatgcaaaaagtaccctttacctgcaaatggacagtctgaggcctgaggacacggccacttattactgtgcaagacggggagcagctatggggggtgttatggatgcctggggtcaaggaacttcagtcactgtctcctcagctgaaacaacagccccatctgtctatccactggctcctggaactgctctca

SEQ ID NO: 18 - protein, 156 aa, Artificial Sequence
  Description of Artificial Sequence Synthetic polypeptide
MDIRLSLAFLVLFIKGVQCEVRLVESGGGLVQPGKSMKLSCVASGFKFSNYYMSWVRQAPAKGLEWVASISDGGGDTFCRDLVKGRFTISRDNAKSTLYLQMDSLRPEDTATYYCARRGAAMGGVMDAWGQGTSVTVSSAETTAPSVYPLAPGTAL

SEQ ID NO: 19 - protein, 10 aa, Artificial Sequence
  Description of Artificial Sequence Synthetic peptide
GFKFSNYYMS

SEQ ID NO: 20 - protein, 17 aa, Artificial Sequence
  Description of Artificial Sequence Synthetic peptide
SISDGGGDTFCRDLVKG

SEQ ID NO: 21 - protein, 15 aa, Artificial Sequence
  Description of Artificial Sequence Synthetic peptide
RGAAMGGVMDAWGQG

SEQ ID NO: 22 - protein, 111 aa, Homo sapiens
PRVAGVPGEAEQPAPELVEVEVGSTALLKCGLSQSQGNLSHVDWFSVHKEKRTLIFRVRQGQGQSEPGEYEQRLSLQDRGATLALTQVTPQDERIFLCQGKRPRSQEYRIQ

SEQ ID NO: 23 - protein, 104 aa, Homo sapiens
PNIQVNPLGIPVNSKEPEEVATCVGRNGYPIPQVIWYKNGRPLKEEKNRVHIQSSQTVESSGLYTLQSILKAQLVKEDKDAQFYCELNYRLPSGNHMKESREVT

SEQ ID NO: 24 - protein, 78 aa, Homo sapiens
PVFYPTEKVWLEVEPVGMLKEGDRVEIRCLADGNPPPHFSISKQNPSTREAEEETTNDNGVLVLEPARKEHSGRYECQ

SEQ ID NO: 25 - protein, 90 aa, Homo sapiens
PQELLVNYVSDVRVSPAAPERQEGSSLTLTCEAESSQDLEFQWLREETDQVLERGPVLQLHDLKREAGGGYRCVASVPSIPGLNRTQLVK

SEQ ID NO: 26 - protein, 81 aa, Homo sapiens
PPWMAFKERKVWVKENMVLNLSCEASGHPRPTISWNVNGTASEQDQDPQRVLSTLNVLVTPELLETGVECTASNDLGKNTS

SEQ ID NO: 27 - protein, 1823 aa, Homo sapiens
MALSSAWRSVLPLWLLWSAACSRAASGDDNAFPFDIEGSSAVGRQDPPETSEPRVALGRLPPAAEKCNAGFFHTLSGECVPCDCNGNSNECLDGSGYCVHCQRNTTGEHCEKCLDGYIGDSIRGAPQFCQPCPCPLPHLANFAESCYRKNGAVRCICNENYAGPNCERCAPGYYGNPLLIGSTCKKCDCSGNSDPNLIFEDCDEVTGQCRNCLRNTTGFKCERCAPGYYGDARIAKNCAVCNCGGGPCDSVTGECLEEGFEPPTGMDCPTISCDKCVWDLTDDLRLAALSIEEGKSGVLSVSSGAAAHRHVNEINATIYLLKTKLSERENQYALRKIQINNAENTMKSLLSDVEELVEKENQASRKGQLVQKESMDTINHASQLVEQAHDMRDKIQEINNKMLYYGEEHELSPKEISEKLVLAQKMLEEIRSRQPFFTQRELVDEEADEAYELLSQAESWQRLHNETRTLFPVVLEQLDDYNAKLSDLQEALDQALNYVRDAEDMNRATAARQRDHEKQQERVREQMEVVNMSLSTSADSLTTPRLTLSELDDIIKNASGIYAEIDGAKSELQVKLSNLSNLSHDLVQEAIDHAQDLQQEANELSRKLHSSDMNGLVQKALDASNVYENIVNYVSEANETAEFALNTTDRIYDAVSGIDTQIIYHKDESENLLNQARELQAKAESSSDEAVADTSRRVGGALARKSALKTRLSDAVKQLQAAERGDAQQRLGQSRLITEEANRTTMEVQQATAPMANNLTNWSQNLQHFDSSAYNTAVNSARDAVRNLTEVVPQLLDQLRTVEQKRPASNVSASIQRIRELIAQTRSVASKIQVSMMFDGQSAVEVHSRTSMDDLKAFTSLSLYMKPPVKRPELTETADQFILYLGSKNAKKEYMGLAIKNDNLVYVYNLGTKDVEIPLDSKPVSSWPAYFSIVKIERVGKHGKVFLTVPSLSSTAEEKFIKKGEFSGDDSLLDLDPEDTVFYVGGVPSNFKLPTSLNLPGFVGCLELATLNNDVISLYNFKHIYNMDPSTSVPCARDKLAFTQSRAASYFFDGSGYAVVRDITRRGKFGQVTRFDIEVRTPADNGLILLMVNGSMFFRLEMRNGYLHVFYDFGFSGGPVHLEDTLKKAQINDAKYHEISIIYHNDKKMILVVDRRHVKSMDNEKMKIPFTDIYIGGAPPEILQSRALRAHLPLDINFRGCMKGFQFQKKDFNLLEQTETLGVGYGCPEDSLISRRAYFNGQSFIASIQKISFFDGFEGGFNFRTLQPNGLLFYYASGSDVFSISLDNGTVIMDVKGIKVQSVDKQYNDGLSHFVISSVSPTRYELIVDKSRVGSKNPTKGKIEQTQASEKKFYFGGSPISAQYANFTGCISNAYFTRVDRDVEVEDFQRYTEKVHTSLYECPIESSPLFLLHKKGKNLSKPKASQNKKGGKSKDAPSWDPVALKLPERNTPRNSHCHLSNSPRAIEHAYQYGGTANSRQEFEHLKGDFGAKSQFSIRLRTRSSHGMIFYVSDQEENDFMTLFLAHGRLVYMFNVGHKKLKIRSQEKYNDGLWHDVIFIRERSSGRLVIDGLRVLEESLPPTEATWKIKGPIYLGGVAPGKAVKNVQINSIYSFSGCLSNLQLNGASITSASQTFSVTPCFEGPMETGTYFSTEGGYVVLDESFNIGLKFEIAFEVRPRSSSGTLVHGHSVNGEYLNVHMKNGQVIVKVNNGIRDFSTSVTPKQSLCDGRWHRITVIRDSNVVQLDVDSEVNHVVGPLNPKPIDHREPVFVGGVPESLLTPRLAPSKPFTGCIRHFVIDGHPVSFSKAALVSGAVSINSCPAA

SEQ ID NO: 28 - protein, 1816 aa, Homo sapiens
MALSSAWRSVLPLWLLWSAACSRAASGDDNAFPFDIEGSSAVGRQDPPETSEPRVALGRLPPAAEKCNAGFFHTLSGECVPCDCNGNSNECLDGSGYCVHCQRNTTGEHCEKCLDGYIGDSIRGAPQFCQPCPCPLPHLANFAESCYRKNGAVRCICNENYAGPNCERCAPGYYGNPLLIGSTCKKCDCSGNSDPNLIFEDCDEVTGQCRNCLRNTTGFKCERCAPGYYGDARIAKNCAVCNCGGGPCDSVTGECLEEGFEPPTGCDKCVWDLTDDLRLAALSIEEGKSGVLSVSSGAAAHRHVNEINATIYLLKTKLSERENQYALRKIQINNAENTMKSLLSDVEELVEKENQASRKGQLVQKESMDTINHASQLVEQAHDMRDKIQEINNKMLYYGEEHELSPKEISEKLVLAQKMLEEIRSRQPFFTQRELVDEEADEAYELLSQAESWQRLHNETRTLFPVVLEQLDDYNAKLSDLQEALDQALNYVRDAEDMNRATAARQRDHEKQQERVREQMEVVNMSLSTSADSLTTPRLTLSELDDIIKNASGIYAEIDGAKSELQVKLSNLSNLSHDLVQEAIDHAQDLQQEANELSRKLHSSDMNGLVQKALDASNVYENIVNYVSEANETAEFALNTTDRIYDAVSGIDTQIIYHKDESENLLNQARELQAKAESSSDEAVADTSRRVGGALARKSALKTRLSDAVKQLQAAERGDAQQRLGQSRLITEEANRTTMEVQQATAPMANNLTNWSQNLQHFDSSAYNTAVNSARDAVRNLTEVVPQLLDQLRTVEQKRPASNVSASIQRIRELIAQTRSVASKIQVSMMFDGQSAVEVHSRTSMDDLKAFTSLSLYMKPPVKRPELTETADQFILYLGSKNAKKEYMGLAIKNDNLVYVYNLGTKDVEIPLDSKPVSSWPAYFSIVKIERVGKHGKVFLTVPSLSSTAEEKFIKKGEFSGDDSLLDLDPEDTVFYVGGVPSNFKLPTSLNLPGFVGCLELATLNNDVISLYNFKHIYNMDPSTSVPCARDKLAFTQSRAASYFFDGSGYAVVRDITRRGKFGQVTRFDIEVRTPADNGLILLMVNGSMFFRLEMRNGYLHVFYDFGFSGGPVHLEDTLKKAQINDAKYHEISIIYHNDKKMILVVDRRHVKSMDNEKMKIPFTDIYIGGAPPEILQSRALRAHLPLDINFRGCMKGFQFQKKDFNLLEQTETLGVGYGCPEDSLISRRAYFNGQSFIASIQKISFFDGFEGGFNFRTLQPNGLLFYYASGSDVFSISLDNGTVIMDVKGIKVQSVDKQYNDGLSHFVISSVSPTRYELIVDKSRVGSKNPTKGKIEQTQASEKKFYFGGSPISAQYANFTGCISNAYFTRVDRDVEVEDFQRYTEKVHTSLYECPIESSPLFLLHKKGKNLSKPKASQNKKGGKSKDAPSWDPVALKLPERNTPRNSHCHLSNSPRAIEHAYQYGGTANSRQEFEHLKGDFGAKSQFSIRLRTRSSHGMIFYVSDQEENDFMTLFLAHGRLVYMFNVGHKKLKIRSQEKYNDGLWHDVIFIRERSSGRLVIDGLRVLEESLPPTEATWKIKGPIYLGGVAPGKAVKNVQINSIYSFSGCLSNLQLNGASITSASQTFSVTPCFEGPMETGTYFSTEGGYVVLDESFNIGLKFEIAFEVRPRSSSGTLVHGHSVNGEYLNVHMKNGQVIVKVNNGIRDFSTSVTPKQSLCDGRWHRITVIRDSNVVQLDVDSEVNHVVGPLNPKPIDHREPVFVGGVPESLLTPRLAPSKPFTGCIRHFVIDGHPVSFSKAALVSGAVSINSCPAA

(19) United States
(12) Patent Application Publication
(10) Pub. No.: US 2020/0165336 A1
(43) Pub. Date: May 28, 2020

(54) MCAM ANTAGONISTS AND METHODS OF TREATMENT

(71) Applicant: PROTHENA BIOSCIENCES LIMITED, Dublin 2 (IE)

(72) Inventors: Kenneth Flanagan, San Francisco, CA (US); Jennifer Johnston, Mill Valley, CA (US); Theodore Yednock, Forest Knolls, CA (US); Jeanne Baker, Redwood City, CA (US)

(73) Assignee: PROTHENA BIOSCIENCES LIMITED, Dublin 2 (IE)

(21) Appl. No.: 16/691,525

(22) Filed: Nov. 21, 2019

Related U.S. Application Data
(63) Continuation of application No. 14/124,620, filed on Jan. 30, 2014, now abandoned.
(60) Provisional application No. 61/493,780, filed on Jun. 6, 2011.
(60) Provisional application No. 61/527,481, filed on Aug. 25, 2011.

(51) Int. Cl. C07K 16/28; C07K 16/18; C07K 16/30

(52) U.S. Cl. CPC C07K 16/2803; C07K 16/18; A61K 2039/505; C07K 16/3092; C07K 16/2896

(57) ABSTRACT

Described herein are MCAM antagonists, including MCAM antagonist antibodies capable of inhibiting the interaction between MCAM and it ligand, a laminin a4 chain, e.g., an ct4 chain of laminin 41 1. These MCAM antagonists, e.g., anti-MCAM antibodies, may be useful to treat neuroinflammatory conditions, for example, multiple sclerosis and Parkinson's disease, by inhibiting the infiltration of MCAM-expressing cells into the central nervous system (CNS), e.g., extravasation of TH 17 cells into the CNS.

### RELATED APPLICATIONS

This application is a continuation of U.S. application Ser. No. 14/124,620 filed Jan. 30, 2014, which is the US national stage entry of PCT/US2012/000274 filed Jun. 6, 2012, which claims the benefit of U.S. Provisional Application No. 61/493,780 filed Jun. 6, 2011 and 61/527,481 filed Aug. 25, 2011, the contents of which are incorporated herein by reference in their entirety.

### SEQUENCE LISTING

This application includes an electronic sequence listing in a file named “439310CON-SEQLST.TXT”, created Nov. 21, 2019 and containing 173,438,615 bytes, which is incorporated by reference.

### FIELD OF THE INVENTION

The present invention concerns melanoma cell adhesion molecule (MCAM) antagonists, including antibodies, capable of inhibiting the interaction between MCAM and its ligand, a laminin α4 chain. These MCAM antagonists, including antagonist antibodies, are useful to treat autoimmune diseases in the central nervous system (CNS), including neuroinflammatory conditions, such as, for example, multiple sclerosis (MS) and Parkinson's disease, by inhibiting the infiltration of MCAM-expressing cells into the CNS, such as, for example by inhibiting the extravasation of TH17 cells into CNS.

### BACKGROUND

A novel subset of CD4+ T cells, termed TH17 cells (T helper 17 cells), has been implicated in the pathogenesis of a number of autoimmune diseases, particularly those neuroinflammatory conditions involving CNS infiltration of T cells, such as multiple sclerosis and the animal model, experimental autoimmune encephalomyelitis (EAE). See, e.g., Cua et al., Nature 421: 744-748 (2003); see also Ivonov et al., Cell 126: 1121-1133 (2006). Much attention on the enhanced pathogenicity of TH17 cells has focused on their ability to secrete a number of select cytokines including IL-17 and IL-22. However, the role of these TH17 cytokines themselves has been called into question, as a conditional knockout of IL-17 is insufficient to affect EAE progression. See, e.g., Haak et al., J. Clin. Invest. 119: 61-69 (2009); see also Kreymborg et al., J. Immunol. 179: 8098-8104 (2007). Although IL-17 affects such vital aspects of EAE as endothelial cell permeability, TH17 cells appear to do more than just produce any one cytokine. The molecular determinants of the pathogenic function of TH17 cells remain elusive.

The pathogenicity of TH17 cells can be partially explained by their unique migration pattern as evidenced by their expression of chemokine receptors. See, e.g., Kim, Inflamm. Allergy Drug Targets 8: 221-228 (2009). It has been established that IL-17 producing cells are enriched within the CCR6+ population of CD4+ T cells, likely conferring a unique migration pattern throughout the vasculature. See, e.g., Acosta-Rodriguez et al., Nat. Immunol. 8:639-646 (2007). In fact, CCR6 expression on T cells is required for T cell migration into the CNS and the progression of EAE. Reboldi et al., Nat. Immunol. 10: 514-523 (2009). A hypothesis has arisen of two waves of T cells, the first a small population of CCR6 expressing TH17 cells that accumulates and recruits a broader second wave of T cells with a more diverse chemokine receptor repertoire. The anatomical site of this infiltration has been suggested to be the choroid plexus due to the constitutive expression of CCL20, a known ligand of CCR6. Ransohoff et al., Nat. Rev. Immunol. 3: 569-581 (2003). The implication has been made that the true pathogenic function of TH17 cells lies in their specific recruitment and infiltration of tissue.

Thus, there is still a need in the art to identify molecules that are involved in the infiltration of TH17 cells into CNS and contribute to their pathogenicity. These molecules can be targets to design therapeutic agents for neuroinflammatory conditions, such as multiple sclerosis (MS) and Parkinson's disease.

### SUMMARY OF THE INVENTION

The present invention concerns MCAM antagonists, e.g., anti-MCAM or anti-laminin α4 chain antibodies, that inhibit the interaction between MCAM and its ligand, laminin α4 chain (e.g., an α4 chain of laminin 411), thereby inhibiting extravasation of TH17 cells into the central nervous system.

TH17 cells play a significant role in the pathogensis of various autoimmune diseases, particularly those displaying neuroinflammatory conditions involving T cells' infiltration into CNS. It has been newly discovered that (1) MCAM is selectively enriched on TH17 cells; and (2) MCAM interacts with a laminin α4 chain, such as, for example, the α4 chain of laminin 411, present in the endothelial basement membrane. An MCAM antagonist, e.g., a monoclonal antibody, capable of inhibiting MCAM's binding to a molecule containing a laminin α4 chain, such as, for example, a laminin 411 molecule, may inhibit the migration of TH17 cells into CNS, and thus can be used as a therapeutic agent to treat diseases displaying neuroinflammatory conditions. MCAM antagonists, such as an MCAM monoclonal antibody or an antigen-binding fragment thereof, may also be useful to treat autoimmune disease, for example, multiple sclerosis, inflammatory bowel disease, psoriasis, and rheumatoid arthritis.

The MCAM antagonists provided herein include, without limitation, monoclonal MCAM antibodies or the antigen-binding fragments thereof that bind to (i) a fragment of MCAM comprising or having the amino acid sequence of position 19 to position 129 of SEQ ID NO: 11 (SEQ ID NO:22); (ii) a fragment of MCAM comprising or having the amino acid sequence of position 139 to position 242 of SEQ ID NO: 11 (SEQ ID NO:23); (iii) a fragment of MCAM comprising or having amino acid sequences shown as SEQ ID NO: 22 and SEQ ID NO: 23. The monoclonal antibody inhibits the binding between MCAM and a laminin α4 chain, e.g., an α4 chain of laminin 411, and/or inhibits TH17 cells' extravasation into central nervous system (CNS). Also provided is a pharmaceutical composition comprising the monoclonal antibody or the antigen-binding fragment thereof. In a preferred embodiment, the laminin α4 chain is an α4 chain of laminin 411.

The monoclonal MCAM antibody can be a chimeric antibody, a humanized antibody, or a human antibody. The present invention provides monoclonal antibodies such as murine antibodies which specifically bind to MCAM. The antibodies of the invention are capable of modulating, e.g., blocking, inhibiting, reducing, antagonizing, neutralizing or otherwise interfering with a biological activity of MCAM. An exemplary monoclonal MCAM antibody or an antigen-binding fragment thereof can comprise a light chain sequence having CDR1, CDR2, and CDR3 as SEQ ID NO: 3, 4, and 5, respectively. The monoclonal MCAM antibody or the antigen-binding fragment thereof may comprise a light chain variable region having the amino acid sequence of SEQ ID NO: 2. The amino acid sequence of the light chain variable region of the monoclonal MCAM antibody or the antigen-binding fragment may differ from the amino acid sequence of SEQ ID NO: 2 by up to one amino acid within the CDR1, CDR2, and CDR3 regions. The amino acid sequence of the light chain variable region of the monoclonal MCAM antibody or the antigen-binding fragment may differ from the amino acid sequence of SEQ ID NO: 2 by multiple amino acids, e.g., up to five amino acids, within the framework regions.

Another exemplary monoclonal MCAM antibody or the antigen-binding fragment thereof can comprise a heavy chain sequence having or comprising CDR1, CDR2, and CDR3 as SEQ ID NO: 8, 9, and 10, respectively. The monoclonal MCAM antibody or the antigen-binding fragment thereof may comprise a heavy chain variable region having the amino acid sequence of SEQ ID NO: 7.

The amino acid sequence of the heavy chain variable region of the monoclonal MCAM antibody or the antigen-binding fragment may differ from the amino acid sequence of SEQ ID NO: 7 by up to one amino acid within the CDR1, CDR2, and CDR3 regions. The amino acid sequence of the heavy chain variable region of the monoclonal MCAM antibody or the antigen-binding fragment may differ from the amino acid sequence of SEQ ID NO: 7 by multiple amino acids, e.g., up to five amino acids, within the framework regions.

A further exemplary monoclonal MCAM antibody or the antigen-binding fragment thereof may comprise (1) a light chain sequence having CDR1, CDR2, and CDR3 as SEQ ID NO: 3, 4, and 5, respectively; and (2) a heavy chain sequence having CDR1, CDR2, and CDR 3 as SEQ ID NO: 8, 9, and 10, respectively.

A method of inhibiting TH17 cells' extravasation into central nervous system is also provided. The method can comprise administering a subject in need thereof with an effective amount of a MCAM antibody or an antigen-binding fragment thereof to inhibit the extravasation into central nervous system. In one embodiment, the subject is suffering from a neuroinflammatory condition. The neuroinflammatory conditions include, for example, multiple sclerosis and Parkinson's disease.

In one aspect, the invention concerns a method for the treatment of a central nervous system (CNS) inflammatory disorder characterized by infiltration of MCAM-expressing cells into the CNS, the method comprising administering to a mammalian subject in need thereof an effective amount of a MCAM antagonist which inhibits binding of MCAM to a laminin α4 chain. In all aspects, the MCAM antagonist preferably is an anti-MCAM or an anti-laminin α4 chain antibody, including antibody fragments. The CNS inflammatory disease preferably is a neuroinflammatory condition, such as, for example, multiple sclerosis (MS) or Parkinson's disease (PD). In a preferred embodiment, the laminin α4 chain is an α4 chain of laminin 411.

### BRIEF DESCRIPTION OF THE DRAWINGS

The accompanying drawings are incorporated into the specification and provide non-limiting illustration of various embodiments. In the drawings:

FIGS. 1A-C depict the presence of MCAM in IL-17-producing human CD4+ cells. FIG. 1A depicts the microarray analysis showing that MCAM is an up-regulated gene in both circulating and activated TH17 cells. FIG. 1B depicts the cell sorting results showing that MCAM exist almost exclusively in a small population of memory T cells (CD45RO+ T cells). FIG. 1C depicts the cell sorting results showing that MCAM is enriched in IL-17-producing human CD4+ T cells.

FIGS. 2A-B depict the surface markers of MCAM expressing T cells. FIG. 2A depicts MCAM expressing T cells as effector memory T cells (CCR6+ while CCR7−). FIG. 2B depicts the integrin expression pattern of MCAM expressing T cells. The majority of MCAM expressing T cells are integrin α4 positive, but are largely integrin β7 negative and β1 positive.

FIGS. 3A-F depict the effects of various cytokines on CD4+/CD45RO+ memory T cells. FIG. 3A depicts the effects of various cytokines on IL-17 production in MCAM positive T cells. FIG. 3B depicts the percentage of cells expressing MCAM following stimulation by various cytokines. FIGS. 3C, 3D, and 3E depict the levels of IL-17 (FIG. 3C), IL-22 (FIG. 3D), and CCL20 (FIG. 3E) in both MCAM positive and MCAM negative cells after stimulations with various cytokines. FIG. 3F depicts the intracellular levels of FOXP3 in both MCAM positive and MCAM negative cells after stimulations with various cytokines.

FIGS. 4A-H depict the identification of laminin 411 as the MCAM ligand. FIG. 4A depicts co-localization of the MCAM ligand and laminin on the choroid plexus of healthy mice. FIG. 4B depicts absence of MCAM staining on the choroid plexus of healthy mice (4′,6-diamidino-2-phenylindole (DAPI) was used as a counterstain). FIG. 4C depicts the presence of MCAM on vascular endothelial cells within healthy mouse brain (DAPI was used as a counterstain). FIG. 4D depicts the expression pattern of the MCAM ligand by staining healthy mouse spinal cord sections with MCAM-Fc protein. FIG. 4E depicts co-localization of the MCAM ligand and laminin on healthy mouse spinal cord. FIG. 4F depicts the extracellular matrix (ECM) localization of the MCAM ligand. CD31 staining was used to show that MCAM staining is exterior to the endothelial cell layer within the vasculature. FIG. 4G depicts the localization of the MCAM ligand within EAE lesions. MCAM-Fc is shown to colocalize with laminin within the endothelial cell basement membrane, but not within the parenchymal basement membrane. FIG. 4H depicts co-localization of the MCAM ligand and laminin 411 (or laminin alpha-4 chain).

FIGS. 5A-C depict specific binding of MCAM antibodies to human and mouse MCAM. FIG. 5B depicts blockage of MCAM-Fc's binding to tissues by MCAM antibodies. FIG. 5C depicts inhibition of the interaction between human MCAM and its ligand laminin 411 by a monoclonal antibody.

FIGS. 6A-B depict the CDRs of the light chain variable region of clone 17 monoclonal antibody. FIG. 6A discloses the nucleic acid sequence encoding the light chain variable region (SEQ ID NO: 1) and the amino acid sequence of the light chain variable region (SEQ ID NO:2), in order of appearance. The three hypervariable regions are also indicated as CDRL1 (SEQ ID NO:3), CDRL2 (SEQ ID NO:4), and CDRL3 (SEQ ID NO:5). FIG. 6B depicts the CDRs of the heavy chain variable region clone 17 monoclonal antibody. FIG. 6B discloses the nucleic acid sequence encoding the heavy chain variable region (SEQ ID NO:6) and the amino acid sequence of the heavy chain variable region (SEQ ID NO:7), in order of appearance. The three hypervariable regions are also indicated as CDRH1 (SEQ ID NO:8), CDRH2 (SEQ ID NO:9), and CDRH3 (SEQ ID NO:10).

FIGS. 7A-B depict absence of MCAM on T cells from naïve mouse. FIG. 7B depicts MCAM expression levels among splenocytes in the presence of various cytokines. Splenocytes were obtained from PLP immunized SJL mice and in vitro restimulated with PLP.

FIGS. 8A-B depict the effects of MCAM blockade on disease progression in a therapeutic model of EAE. After EAE symptoms appeared, PLP-immunized mice were treated intraperitoneally with (1) anti-MCAM antibody (clone 15) at 10 mg/kg body weight, (2) the isotype control (Bioxcell) at 10 mg/kg body weight, and (3) PBS every day thereafter. The disease progression (FIG. 8A) and body weights (FIG. 8B) were monitored every 2-3 days. Data represent the mean of 15 mice±sem (standard error of the mean).

FIGS. 9A-B depict the CDRs of the light chain variable region of clone 15 monoclonal antibody. FIG. 9A discloses the nucleic acid sequence encoding the light chain variable region (SEQ ID NO: 12 and the amino acid sequence of the light chain variable region (SEQ ID NO: 13), in order of appearance. The three hypervariable regions are also indicated as CDRL1 (SEQ ID NO: 14), CDRL2 (SEQ ID NO: 15), and CDRL3 (SEQ ID NO:16). FIG. 9B depicts the CDRs of the heavy chain variable region clone 15 monoclonal antibody. FIG. 9B discloses the nucleic acid sequence encoding the heavy chain variable region (SEQ ID NO: 17) and the amino acid sequence of the heavy chain variable region (SEQ ID NO: 18), in order of appearance. The three hypervariable regions are also indicated as CDRH1 (SEQ ID NO: 19), CDRH2 (SEQ ID NO:20), and CDRH3 (SEQ ID NO:21).

FIGS. 10A-B depict the results of a domain binding test for MCAM antibodies.

FIGS. 11A-B depict the amino acid sequence (A) (SEQ ID NO: 11—Accession No. CAA48332) and structure (B) for human MCAM. In FIG. 11A, the amino acid residue positions corresponding to the five immunoglobulin domains of human MCAM are as follows—1: amino acid residues 19-129; 2: amino acid residues 139-242; 3: amino acid residues 244-321; 4: amino acid residues 335-424; and 5: amino acid residues 430-510) (SEQ ID NOS:22-26), which are also depicted schematically in FIG. 11B.

FIGS. 12A-B show the amino acid sequences for two α4-chain isoforms of human laminin 411. FIG. 12A shows the amino acid sequence corresponding to GenBank Accession No. NP001098676 (SEQ ID NO: 27) and FIG. 12B shows the amino acid sequence corresponding to GenBank Accession No. NP001098677 (SEQ ID NO: 28).

### DETAILED DESCRIPTION

### 1. Definitions and Abbreviations

### 1.1. Definitions

An “individual” or “subject” as used herein may be any of mammalian animals (e.g., domesticated animals), including human, dog, cat, cattle, horse, goat, pig, swine, sheep, monkey, guinea pig, rat, and mouse. In one embodiment, the individual or subject can be a human.

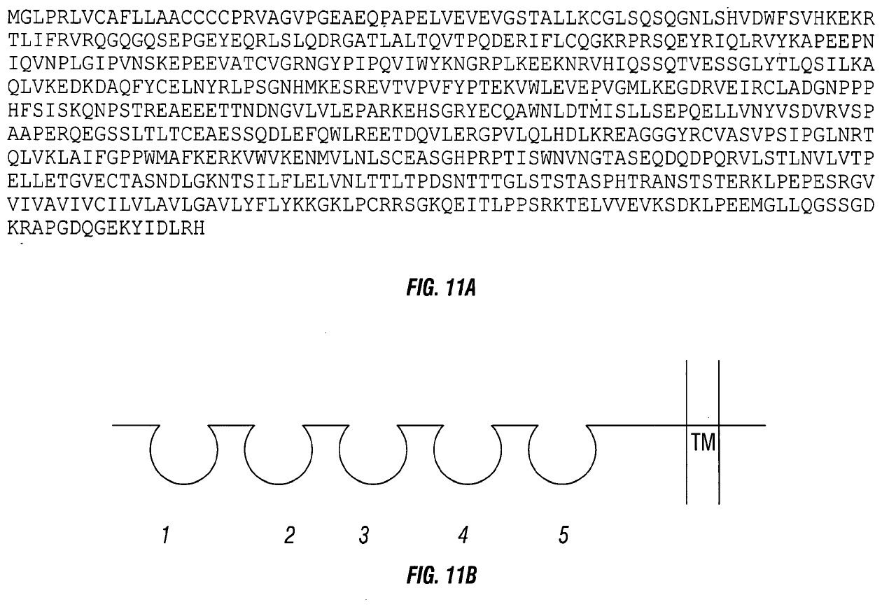

“MCAM” (melanoma cell adhesion molecule, also known as CD146 and MUC18) refers to a cell surface glycoprotein belonging to the immunoglobulin superfamily involved in cell adhesion, and in cohesion of the endothelial monolayer at intercellular junctions in vascular tissue. It also promotes tumor progression of many cancers including melanoma and prostate cancer. It is known to interact in a homotypic/homophilic manner and may also bind to other ligands. The human MCAM has the amino acid sequence of SEQ ID NO: 11 (FIG. 11A), which includes five immunoglobulin domains (1: amino acid residues 19-129; 2: amino acid residues 139-242; 3: amino acid residues 244-321; 4: amino acid residues 335-424; and 5: amino acid residues 430-510) shown as SEQ ID NOS:22-26, which are also depicted schematically in FIG. 11B.

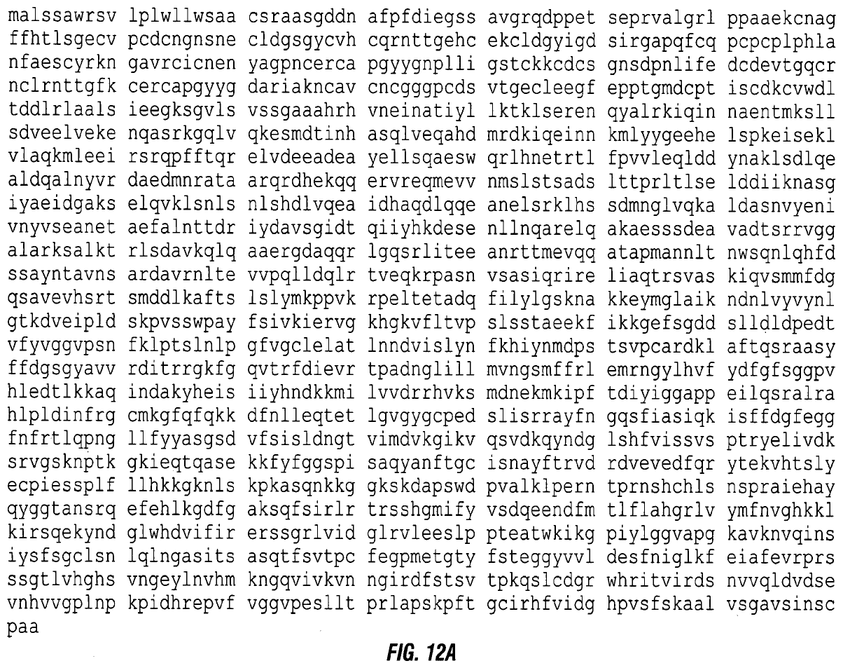

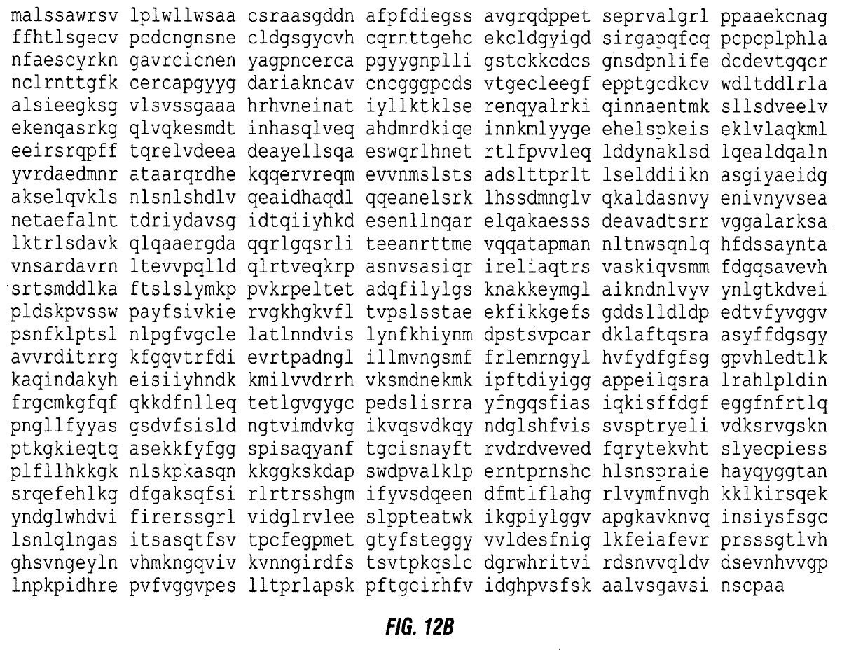

A “laminin α4 chain” refers to one of the polypeptide chains found in laminin molecules, which are expressed in the basal lamina (of the basement membrane), a protein network foundation for most cells and organs. Laminins are known to bind to cell membranes through plasma membrane molecules and contribute to cell attachment. The laminin α4 chain typically forms a complex with a laminin β-chain, and a laminin γ-chain. The laminin α4 chain is found in numerous laminin molecules including, without limitation, laminin 411 (laminin 8 or α4β1γ1); laminin 421 (laminin 9 or α4β2γ1), and laminin 423 (laminin 14 or α4β2γ3). There are two main isoforms of the human laminin α4-chain: GenBank Accession Nos. NP001098676 and NP001098677 as shown in FIG. 12A-B (amino acid sequences SEQ ID NOS:27-28). “Laminin 411” refers to a trimeric polypeptide complex made up of three polypeptide subunits or chains: α4-chain, a β1-chain, and a γ1-chain.

The term “antagonist” is used in the broadest sense, and includes any molecule that partially or fully blocks, inhibits, or neutralizes a qualitative biological activity of an MCAM polypeptide. For the purpose of the present invention, the biological activity preferably is the ability to inhibit the ability of MCAM (i) to specifically bind its ligand: a laminin α4 chain, e.g., the α4 chain of laminin 411; and/or (ii) to facilitate an MCAM-expressing cell, e.g., a TH17 cell, to infiltrate into or migrate to a subject's tissue. Antagonists of MCAM can be identified, for example, based upon their ability to inhibit or block the specific binding of MCAM to its ligand: a laminin α4 chain, e.g., the α4 chain of laminin 411. MCAM antagonists specifically include, without limitation, antibodies (e.g., antagonist or neutralizing antibodies), including chimeric, humanized and human antibodies and their functional fragments, small molecules, ribozymes, aptamers, peptides, and nucleic acids that encode polypeptide antagonists or antagonist antibodies.

The term “MCAM antagonist antibody” refers to an antibody which inhibits or neutralizes the activity of MCAM. Such an antibody specifically binds to a polypeptide target involved in the infiltration of an MCAM-expressing cell into the CNS, e.g., MCAM or a laminin α4 chain (e.g., the α4 chain of laminin 411).

A “blocking” antibody, “neutralizing” antibody, or “antagonist” antibody is one which inhibits or reduces a biological activity of the antigen it binds. Such antibodies may substantially or completely inhibit the biological activity of the antigen.

The terms “specifically binds” or “binds specifically” as used herein means that one member of a specific binding pair will not show any statistically significant binding to molecules other than its specific binding partner. A binding partner may show at least 1000 times the affinity of binding (measured as an apparent association constant) for its specific binding pair partner than a non-specific binding partner. For example, antibodies that bind to MCAM with a binding affinity of 10⁷ mole/L or more, typically 10⁸ mole/L or more, are said to bind specifically to MCAM.

The terms “biological activity” and “biologically active” with regard to MCAM refer to its ability to specifically bind its ligand (a laminin α4 chain, e.g., the (α4 chain of laminin 411) and/or to facilitate the infiltration of MCAM-expressing cells, e.g., TH17 cells, into the CNS.

The term an “MCAM-expressing cell” refers to a cell of the immune system that expresses MCAM. For example, MCAM expression is enriched on memory T lymphocytes, e.g., TH17 cells.

The term “binding molecule” as used herein refers to a molecule that specifically binds to a target. The term specifically includes, without limitation, antibodies and antibody fragments (e.g. those comprising one or more of the CDRs described herein), and peptide and non-peptide small molecules.

“Antibodies” (Abs) and “immunoglobulins” (Igs) are glycoproteins having some common structural characteristics. While antibodies exhibit binding specificity to a specific antigen, immunoglobulins include both antibodies and other antibody-like molecules which lack antigen specificity. Polypeptides of the latter kind can be, for example, produced at low levels by the lymph system and at increased levels by myelomas.

The term “antibody” used herein may encompass intact monoclonal antibodies, polyclonal antibodies, multispecific antibodies (e.g. bispecific antibodies) formed from at least two intact antibodies, and antibody fragments, so long as they exhibit the desired biological activity. The term “antigen-binding fragment” of an antibody refers to a portion of the full-length immunoglobulin molecule that specifically binds to the antigen. An antigen-binding fragment of an antibody thus includes an antigen-binding heavy chain, light chain, heavy chain-light chain dimer, Fab fragment, F(ab′)2 fragment, Fv fragment, single chain Fv (scFv), diabodies, linear antibodies, and multispecific antibodies formed from antibody fragment(s).

The term “monoclonal antibody” as used herein refers to an antibody from a population of substantially homogeneous antibodies, i.e., the individual antibodies comprising the population are substantially similar and bind the same epitope(s), except for possible variants that may arise during production of the monoclonal antibody, such variants generally being present in minor amounts. Such monoclonal antibody typically includes an antibody comprising a variable region that binds a target, wherein the antibody was obtained by a process that includes the selection of the antibody from a plurality of antibodies. For example, the selection process can be the selection of a unique clone from a plurality of clones, such as a pool of hybridoma clones, phage clones or recombinant DNA clones. It should be understood that the selected antibody can be further altered, for example, to improve affinity for the target, to humanize the antibody, to improve its production in cell culture, to reduce its immunogenicity in vivo, to create a multispecific antibody, etc., and that an antibody comprising the altered variable region sequence is also a monoclonal antibody of this invention. In addition to their specificity, the monoclonal antibody preparations are advantageous in that they are typically uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including the hybridoma method (e.g., Kohler et al., Nature, 256:495 (1975); Harlow et al., Antibodies: A Laboratory Manual, (Cold Spring Harbor Laboratory Press, 2nd ed. 1988); Hammerling et al., in: Monoclonal Antibodies and T-Cell Hybridomas 563-681, (Elsevier, N. Y., 1981), recombinant DNA methods (see, e.g., U.S. Pat. No. 4,816,567), phage display technologies (see, e.g., Clackson et al., Nature, 352:624-628 (1991); Marks et al., J. Mol. Biol., 222:581-597 (1991); Sidhu et al., J. Mol. Biol. 338(2):299-310 (2004); Lee et al., J. Mol. Biol. 340(5):1073-1093 (2004); Fellouse, Proc. Nat. Acad. Sci. USA 101(34):12467-12472 (2004); and Lee et al. J. Immunol. Methods 284(1-2):119-132 (2004) and technologies for producing human or human-like antibodies from animals that have parts or all of the human immunoglobulin loci or genes encoding human immunoglobulin sequences (see, e.g., WO98/24893, WO/9634096, WO/9633735, and WO/91 10741, Jakobovits et al., Proc. Natl. Acad. Sci. USA, 90:2551 (1993); Jakobovits et al., Nature, 362:255-258 (1993); Bruggemann et al., Year in Immune, 7:33 (1993); U.S. Pat. Nos. 5,545,806, 5,569,825, 5,591,669 (all of GenPharm); 5,545,807; WO 97/17852, U.S. Pat. Nos. 5,545,807; 5,545,806; 5,569,825; 5,625,126; 5,633,425; and 5,661,016, and Marks et al., Bio/Technology, 10: 779-783 (1992); Lonberg et al., Nature, 368: 856-859 (1994); Morrison, Nature, 368: 812-813 (1994); Fishwild et al., Nature Biotechnology, 14: 845-851 (1996); Neuberger, Nature Biotechnology, 14: 826 (1996); and Lonberg and Huszar, Intern. Rev. Immunol., 13: 65-93 (1995).

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA, 81:6851-6855 (1984)). Chimeric antibodies of interest herein include “primatized” antibodies comprising variable domain antigen-binding sequences derived from a non-human primate (e.g. Old World Monkey, Ape etc) and human constant region sequences, as well as “humanized” antibodies.

“Humanized” forms of non-human (e.g., rodent) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

An “intact antibody” herein is one which comprises two antigen binding regions, and an Fc region. Preferably, the intact antibody has a functional Fc region.

An “antibody (or any other binding molecule) that binds to the same epitope” as a reference antibody (or any other binding molecule) refers to an antibody (or any other binding molecule) that blocks binding of the reference antibody (or any other binding molecule) to its antigen in a competition assay by 50% or more, and conversely, the reference antibody (or any other binding molecule) blocks binding of the antibody to its antigen in a competition assay by 50% or more.

An “affinity matured” antibody is one with one or more alterations in one or more hypervariable regions thereof which result an improvement in the affinity of the antibody for antigen, compared to a parent antibody which does not possess those alteration(s). Preferred affinity matured antibodies will have nanomolar or even picomolar affinities for the target antigen. Affinity matured antibodies are produced by procedures known in the art. Marks et al. Bio/Technology 10:779-783 (1992) describes affinity maturation by VH and VL domain shuffling. Random mutagenesis of CDR and/or framework residues is described by: Barbas et al. Proc Nat. Acad. Sci, USA 91:3809-3813 (1994); Schier et al. Gene 169:147-155 (1995); Yelton et al. J. Immunol. 155:1994-2004 (1995); Jackson et al., J. Immunol. 154(7):3310-9 (1995); and Hawkins et al, J. Mol. Biol. 226:889-896 (1992).

The “light chains” of antibodies from any vertebrate species can be assigned to one of two clearly distinct types, called κ and λ, based on the amino acid sequences of their constant domains. Depending on the amino acid sequence of the constant domain of their heavy chains, intact antibodies can be assigned to different “classes.” There are five major classes of intact antibodies: IgA, IgD, IgE, IgG, and IgM, and several of these may be further divided into “subclasses” (isotypes), e.g., IgG1, IgG2, IgG3, IgG4, IgA, and IgA2. The heavy-chain constant domains that correspond to the different classes of antibodies are called α, δ, ε, γ, and μ respectively. The subunit structures and three-dimensional configurations of different classes of immunoglobulins are well known.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called complementarity-determining regions (CDRs) or hypervariable regions (HVRs) both in the light-chain and heavy-chain variable domains. The more highly conserved portions of variable domains are called the framework (FR). The variable domains of native heavy and light chains each comprise four FR regions, largely adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen-binding site of antibodies. The constant domains are not involved directly in binding an antibody to an antigen, but exhibit various effector functions, such as participation of the antibody in antibody-dependent cellular toxicity.

“Fv” is the minimum antibody fragment which contains a complete antigen-recognition and binding site. In a two-chain Fv species, this region consists of a dimer of one heavy- and one light-chain variable domain in tight, non-covalent association. In a single-chain Fv species, one heavy- and one light-chain variable domain can be covalently linked by a flexible peptide linker such that the light and heavy chains can associate in a “dimeric” structure analogous to that in a two-chain Fv species. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

“Hypervariable region” or “HVR” refers to the amino acid residues of an antibody that are responsible for antigen-binding. The hypervariable region generally comprises amino acid residues from a “complementarity determining region” or “CDR” (Kabat et al., SEQUENCES OF PROTEINS OF IMMUNOLOGICAL INTEREST, 5^{th} Ed. Public Health Service, National Institutes of Health, Bethesda, Md. (1991)) and/or those residues from a “hypervariable loop” (Chothia and Lesk, J. Mol. Biol. 196: 901-917 (1987)).

The term “complementarity determining regions” or “CDRs” when used herein refers to parts of immunological receptors that make contact with a specific ligand and determine its specificity. The CDRs of immunological receptors are the most variable part of the receptor protein, giving receptors their diversity, and are carried on six loops at the distal end of the receptor's variable domains, three loops coming from each of the two variable domains of the receptor.

The term “epitope” is used to refer to binding sites for (monoclonal or polyclonal) antibodies on protein antigens. Typically, an epitope refers to a unit of structure conventionally bound by an immunoglobulin VH-VL pair. Epitopes define the minimum binding site for an antibody, and thus represent the target of specificity of an antibody. Epitopes can be linear or conformational, and can be as small as three amino acids.

A “small molecule” is defined herein to have a molecular weight below about 600, preferably below about 1000 daltons. Generally, a small molecule is a non-peptide small organic molecule.

The terms “affinity”, “binding affinity” and “K_{d}” refer to the equilibrium dissociation constant (expressed in units of concentration) associated with each MCAM binding molecule-target complex, such as between an anti-MCAM antibody and MCAM. The binding affinity is directly related to the ratio of the off-rate constant (generally reported in units of inverse time, e.g., seconds⁻¹) to the on-rate constant (generally reported in units of concentration per unit time, e.g., molar/second). The binding affinity may be determined by, for example, an ELISA assay, kinetic exclusion assay or surface plasmon resonance. It is noted that certain epitopes can occur repetitively (multivalent) on a cell surface and that the dissociation constant (koff) for the binding of an antibody to a repetitive epitope may be greatly diminished over the dissociation constant for the reaction of the same antibody with the corresponding ligand in univalent form. The diminished dissociation constant arises because when one antibody-ligand bond dissociates, other bonds hold the bivalent (or multivalent) antibody to the multivalent ligand, allowing the dissociated bond to form again. The dissociation constant for the reaction between bivalent (or multivalent) Ab and multivalent ligand has been termed the functional affinity to contrast it with intrinsic affinity, which is the association constant for an antibodies representative individual site.

The terms “dissociation”, “dissociation rate” and “k_{off}” as used herein, are intended to refer to the off rate constant for dissociation of a binding molecule, such as an antibody, from the binding molecule/target, e.g. antibody/antigen complex.

The terms “association”, “association rate” and “kₒₙ” as used herein, are intended to refer to the on rate constant for association of a binding molecule with a target, such as an antibody with an antigen, to form a complex.

The terms “effective concentration” and “EC₅₀” as used herein, are intended to refer to the concentration of a binding molecule (e/g/antibody) capable of interacting with sufficient quantities of target molecules to produce an effect on approximately 50% of the treated cells.

As used herein, “treatment” (and grammatical variations thereof such as “treat” or “treating”) refers to clinical intervention in an attempt to alter the natural course of the individual being treated, and can be performed either for prophylaxis/prevention, or during the course of clinical pathology. The term refers to both therapeutic treatment and prophylactic or preventative measures, wherein the object is to prevent or slow down (lessen) an undesired physiological change or disorder. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

“Chronic” administration refers to administration of the agent(s) in a continuous mode as opposed to an acute mode, so as to maintain the desired effect for an extended period of time. “Intermittent” administration is treatment that is not consecutively done without interruption, but rather is cyclic in nature.

An “effective amount” refers to an amount effective, at dosages and for periods of time necessary, to achieve the desired prophylactic or therapeutic result. An effective amount refers to the amount of active compound or pharmaceutical agent that elicits the biological or medicinal response in a tissue, system, animal, individual or human that is being sought by a researcher, veterinarian, medical doctor or other clinician, which includes one or more of the following:

(A) preventing the disease; for example, preventing an inflammatory disease, such as a neuroinflammatory disease, condition or disorder in an individual that may be predisposed to the disease, condition or disorder but does not yet experience or display the pathology or symptoms of the disease,

(B) inhibiting the disease; for example, inhibiting an inflammatory disease, such as a neuroinflammatory disease, condition or disorder in an individual that is experiencing or displaying the pathology or symptoms of the disease, condition or disorder (i.e., arresting further development of the pathology and/or symptoms), and

(C) ameliorating the disease; for example, ameliorating an inflammatory disease, such as a neuroinflammatory disease, condition or disorder in an individual that is experiencing or displaying the pathology or symptoms of the disease, condition or disorder (i.e., reversing the pathology and/or symptoms).

Unless defined otherwise, all technical and scientific terms used herein have the same meaning as commonly understood by one of ordinary skill in the art. In some cases, terms with commonly understood meanings are defined herein for clarity and/or for ready reference, and the inclusion of such definitions herein should not necessarily be construed to represent a substantial difference over what is generally understood in the art. The techniques and procedures described or referenced herein are generally well understood and commonly employed using conventional methodology by those skilled in the art, such as, for example, the widely utilized molecular cloning methodologies described in Sambrook et al., Molecular Cloning: A Laboratory Manual 2nd. edition (1989) Cold Spring Harbor Laboratory Press, Cold Spring Harbor, N. Y. As appropriate, procedures involving the use of commercially available kits and reagents are generally carried out in accordance with manufacturer defined protocols and/or parameters unless otherwise noted. Before the present methods, kits and uses therefore are described, it is to be understood that this invention is not limited to the particular methodology, protocols, cell lines, animal species or genera, constructs, and reagents described as such may, of course, vary. It is also to be understood that the terminology used herein is for the purpose of describing particular embodiments only, and is not intended to limit the scope of the present invention which will be limited only by the appended claims.

It must be noted that as used herein, the singular forms “a”, “and”, and “the” include plural referents unless the context clearly dictates otherwise. Thus, for example, reference to “an antibody” includes a plurality of such antibodies and reference to “the dosage” includes reference to one or more dosages and equivalents thereof known to those skilled in the art, and so forth. Throughout this specification and claims, the word “comprise,” or variations such as “comprises” or “comprising,” will be understood to imply the inclusion of a stated integer or group of integers but not the exclusion of any other integer or group of integers.

### Abbreviations

Abs antibodies

CDR complementarity determining region

CFA complete Freund's adjuvant

CFSE carboxyfluorescein succinimidyl ester

CNS central nervous system

DAPI 4′,6-diamidino-2-phenylindole

DN dopamine-containing neuron

EAE experimental autoimmune encephalomyelitis

ECM extracellular matrix

FACS fluorescence Activated cell sorting

FR Framework Region

IFA incomplete Freund's adjuvant

Igs immunoglobulins

MCAM melanoma cell adhesion molecule

MOG myelin oligodendrocyte glycoprotein (MOG)

MS multiple sclerosis

PD Parkinson's disease

PMA phorbol myristate acetate

### 2. MCAM

MCAM (melanoma cell adhesion molecule) is a cell-surface glycoprotein originally identified as a melanoma antigen, whose expression is associated with tumor progression and the development of metastatic potential. MCAM is a 113 kDA cell surface integral membrane glycoprotein composed of a signal peptide, five immunoglobulin-like domains (1, 2, 3, 4, and 5; or V—V-C2-C2-C2), a transmembrane region, and a short cytoplasmic tail. See, e.g., Lehmann et al., Proc. Nat'l Acad. Sci. USA 86: 9891-9895 (1989) and FIG. 11B. MCAM is a member of the immunoglobulin superfamily and has significant sequence homology to a number of cell adhesion molecules of the Ig superfamily, including BEN (Pourquie et al., Proc. Nat'l Acad. Sci. USA 89: 5261-5265 (1992)), neural-cell adhesion molecule (N-CAM) (Owens et al., Proc. Nat'l Acad. Sci. USA 84: 294-298 (1987)), myelin-associated glycoprotein (MAG) (Lai et al., Proc. Nat'l Acad. Sci. USA 84: 4337-4341 (1987)), deleted in colorectal cancer protein (DCC) (Hedrick et al., Genes Devel. 8: 1174-1183 (1994)), and gicerin (Taira et al., Neuron 12: 861-872 (1994)). The expression of MCAM has been detected in relatively limited spectrum of normal human tissues and in a variety of malignant neoplasms. In normal adult tissues, MCAM is expressed on endothelial cells, smooth muscle cells (Shih et al., Lab. Invest. 75: 377-388 (1996); Sers et al., Cancer Res. 54: 5689-5694 (1994)), a subpopulation of activated T lymphocytes (Pickl et al., J. Immunol. 158: 2107-2115 (1997)), and intermediate trophoblasts (Shih et al., supra). MCAM is also expressed on a variety of malignant neoplasms including smooth muscle neoplasms (Leiomyomas and leiomyosarcomas), tumors of vascular origin (angiosarcomas and Kaposi's sarcomas), placental site trophoblastic tumors, choriocarcinomas, and melanomas (Shih et al., Clinical Cancer Res. 2: 569-575 (1996); Holzmann et al., Int. J. Cancer 39: 466-471 (1987)). The expression of MUC18 correlates directly with the metastatic potential of human melanoma cells (Bar-Eli, Cancer Metastasis, 18: 377-385 (1999)).

A number of studies have identified MCAM as a marker of tumor progression and metastasis in melanomas. The expression of MCAM is absent in normal melanocytes and benign nevi but prominent on many primary melanomas and in most metastatic lesions (Lehmann et al., supra; Shih et al., supra). MCAM expression correlates well with tumor vertical thickness and metastasis formation, and greater than 80% of metastatic lesions express MCAM (Lehmann et al., supra; Xie et al., Cancer Res. 57: 2295-2303 (1997); and Shih et al., supra). Modulators of MCAM have been generated to treat melanomas. See, e.g., U.S. Pat. No. 7,067,131. Recently, MCAM modulation has been suggested to identify and select inflammatory cytokine-secreting T cells or their precursors to treat various inflammatory conditions. See, e.g., U.S. Published Patent Application No. 2011/0014183.

### 3. Neuroinflammatory Conditions, Multiple Sclerosis, and Parkinson Disease

A neuroinflammatory condition refers to a condition associated with inflammation of the nervous system, in an embodiment the central nervous system (CNS), and which is associated with cell/tissue damage. It is typically characterized by, for example, increased glial activation, increased pro-inflammatory cytokine/chemokine levels (e.g., TNFα, INFγ, IL-β), increased blood-brain-barrier permeability, and/or increased immune cell (e.g., leukocyte) recruitment/invasion to the CNS. It may refer to, for example, chronic neuroinflammation, such as an inflammation associated with chronic activation of cells of the immune system (i.e., autoimmune-associated neuroinflammation). Such chronic neuroinflammation can be observed in, for example, multiple sclerosis (MS). Additionally, Parkinson's disease (PD) is a neurodegenerative disease displaying neuroinflammation, for example, activated microglia and infiltrating T cells.

Multiple sclerosis, as a progressive neurological autoimmune disease, results from chronic, pathological inflammation (Yednock et al., Nature 356: 63-66 (1992); Baron et al., J Exp. Med. 177: 57-68 (1993)). MS affects an estimated 250,000 to 350,000 people in the United States. Multiple sclerosis is thought to be the result of a specific autoimmune reaction wherein certain leukocytes attack and initiate the destruction of myelin, the insulating sheath covering nerve fibers. The onset of MS may be dramatic or so mild as to not cause a patient to seek medical attention. The most common symptoms include weakness in one or more limbs, visual blurring due to optic neuritis, sensory disturbances, diplopia, and ataxia. The course of disease may be stratified into three general categories: (1) relapsing MS, (2) chronic progressive MS, and (3) inactive MS.

Relapsing MS is generally characterized by recurrent attacks of neurologic dysfunction. MS attacks generally evolve over days to weeks and may be followed by complete, partial, or no recovery. Recovery from attacks generally occurs within weeks to several months from the peak of symptoms, although rarely some recovery may continue for 2 or more years.

Chronic progressive MS results in gradually progressive worsening without periods of stabilization or remission. This form develops in patients with a prior history of relapsing MS, although in 20% of patients, no relapses can be recalled. Acute relapses also may occur during the progressive course of MS.

A third form is inactive MS. Inactive MS is characterized by fixed neurologic deficits of variable magnitude. Most patients with inactive MS have an earlier history of relapsing MS. The course of MS is also dependent on the age of the patient. For example, favorable prognostic factors include early onset (excluding childhood), a relapsing course and little residual disability 5 years after onset. By contrast, poor prognosis is associated with a late age of onset (i.e., age 40 or older) and a progressive course. These variables are interdependent, since chronic progressive MS tends to begin at a later age that relapsing MS. Disability from chronic progressive MS is usually due to progressive paraplegia or quadriplegia in individual patients.

Parkinson's disease (PD) is a progressive neurodegenerative disease displaying primary clinical features of motor abnormalities, e.g., resting tremor, bradykinesia, and rigidity. PD is characterized by the loss of dopamine-containing neuron (DN) cells in the substantia nigra parts compacta (Forno, J. Neurophthol. Exp. Neurol. 55: 259-272 (1996)). One of the hallmarks of PD is neuroinflammation characterized by activated microglia and infiltrating T cells. Although studies have suggested various mechanisms for PD, such as mitochonodrial dysfunction, oxidative stress, and impairment of protein degradation machinery, the cause of PD remains elusive (Dauer et al., Neuron 39: 889-909 (2003)). Recent findings have indicated that both innate and adaptive immunity may play important roles in the pathogenesis of PD (Stone et al., Antioxid. Redox. Signal. 11: 2151-2166 (2009)). Particularly, it has been shown in the animal model of PD that both activated microglia and T lymphocytes contribute significantly to neurodegeneration. See, e.g., Brochard et al., J. Clin. Invest. 119: 182-192 (2009). It has been hypothesized that CD4 positive T cells (e.g., proinflammatory T17 cells) mediate cytotoxicity by activating microglia in PD and/or exert a direct toxic effect on substanitia nigra DNs (Appel, J. Clin. Invest. 119: 13-15 (2009)).

### 4. Autoimmune Diseases

An autoimmune disease herein is a disease or disorder arising from and directed against an individual's own tissues or a co-segregate or manifestation thereof or resulting condition therefrom. Examples of autoimmune diseases or disorders include, but are not limited to arthritis (rheumatoid arthritis such as acute arthritis, chronic rheumatoid arthritis, gout or gouty arthritis, acute gouty arthritis, acute immunological arthritis, chronic inflammatory arthritis, degenerative arthritis, type II collagen-induced arthritis, infectious arthritis, Lyme arthritis, proliferative arthritis, psoriatic arthritis, Still's disease, vertebral arthritis, and juvenile-onset rheumatoid arthritis, osteoarthritis, arthritis chronica progrediente, arthritis deformans, polyarthritis chronica primaria, reactive arthritis, and ankylosing spondylitis), inflammatory hyperproliferative skin diseases, psoriasis such as plaque psoriasis, gutatte psoriasis, pustular psoriasis, and psoriasis of the nails, atopy including atopic diseases such as hay fever and Job's syndrome, dermatitis including contact dermatitis, chronic contact dermatitis, exfoliative dermatitis, allergic dermatitis, allergic contact dermatitis, dermatitis herpetiformis, nummular dermatitis, seborrheic dermatitis, non-specific dermatitis, primary irritant contact dermatitis, and atopic dermatitis, x-linked hyper IgM syndrome, allergic intraocular inflammatory diseases, urticaria such as chronic allergic urticaria and chronic idiopathic urticaria, including chronic autoimmune urticaria, myositis, polymyositis/dermatomyositis, juvenile dermatomyositis, toxic epidermal necrolysis, scleroderma (including systemic scleroderma), sclerosis such as systemic sclerosis, multiple sclerosis (MS) such as spino-optical MS, primary progressive MS (PPMS), and relapsing remitting MS (RRMS), progressive systemic sclerosis, atherosclerosis, arteriosclerosis, sclerosis disseminata, ataxic sclerosis, neuromyelitis optica (NMO), inflammatory bowel disease (IBD) (for example, Crohn's disease, autoimmune-mediated gastrointestinal diseases, colitis such as ulcerative colitis, colitis ulcerosa, microscopic colitis, collagenous colitis, colitis polyposa, necrotizing enterocolitis, and transmural colitis, and autoimmune inflammatory bowel disease), bowel inflammation, pyoderma gangrenosum, erythema nodosum, primary sclerosing cholangitis, respiratory distress syndrome, including adult or acute respiratory distress syndrome (ARDS), meningitis, inflammation of all or part of the uvea, iritis, choroiditis, an autoimmune hematological disorder, rheumatoid spondylitis, rheumatoid synovitis, hereditary angioedema, cranial nerve damage as in meningitis, herpes gestationis, pemphigoid gestationis, pruritis scroti, autoimmune premature ovarian failure, sudden hearing loss due to an autoimmune condition, IgE-mediated diseases such as anaphylaxis and allergic and atopic rhinitis, encephalitis such as Rasmussen's encephalitis and limbic and/or brainstem encephalitis, uveitis, such as anterior uveitis, acute anterior uveitis, granulomatous uveitis, nongranulomatous uveitis, phacoantigenic uveitis, posterior uveitis, or autoimmune uveitis, glomerulonephritis (GN) with and without nephrotic syndrome such as chronic or acute glomerulonephritis such as primary GN, immune-mediated GN, membranous GN (membranous nephropathy), idiopathic membranous GN or idiopathic membranous nephropathy, membrano- or membranous proliferative GN (MPGN), including Type I and Type II, and rapidly progressive GN, proliferative nephritis, autoimmune polyglandular endocrine failure, balanitis including balanitis circumscripta plasmacellularis, balanoposthitis, erythema annulare centrifugum, erythema dyschromicum perstans, eythema multiform, granuloma annulare, lichen nitidus, lichen sclerosus et atrophicus, lichen simplex chronicus, lichen spinulosus, lichen planus, lamellar ichthyosis, epidermolytic hyperkeratosis, premalignant keratosis, pyoderma gangrenosum, allergic conditions and responses, allergic reaction, eczema including allergic or atopic eczema, asteatotic eczema, dyshidrotic eczema, and vesicular palmoplantar eczema, asthma such as asthma bronchiale, bronchial asthma, and auto-immune asthma, conditions involving infiltration of T cells and chronic inflammatory responses, immune reactions against foreign antigens such as fetal A-B-O blood groups during pregnancy, chronic pulmonary inflammatory disease, autoimmune myocarditis, leukocyte adhesion deficiency, lupus, including lupus nephritis, lupus cerebritis, pediatric lupus, non-renal lupus, extra-renal lupus, discoid lupus and discoid lupus erythematosus, alopecia lupus, systemic lupus erythematosus (SLE) such as cutaneous SLE or subacute cutaneous SLE, neonatal lupus syndrome (NLE), and lupus erythematosus disseminatus, juvenile onset (Type I) diabetes mellitus, including pediatric insulin-dependent diabetes mellitus (IDDM), adult onset diabetes mellitus (Type II diabetes), autoimmune diabetes, idiopathic diabetes insipidus, diabetic retinopathy, diabetic nephropathy, diabetic large-artery disorder, immune responses associated with acute and delayed hypersensitivity mediated by cytokines and T-lymphocytes, tuberculosis, sarcoidosis, granulomatosis including lymphomatoid granulomatosis, Wegener's granulomatosis, agranulocytosis, vasculitides, including vasculitis, large-vessel vasculitis (including polymyalgia rheumatica and giant-cell (Takayasu's) arteritis), medium-vessel vasculitis (including Kawasaki's disease and polyarteritis nodosa/periarteritis nodosa), microscopic polyarteritis, immunovasculitis, CNS vasculitis, cutaneous vasculitis, hypersensitivity vasculitis, necrotizing vasculitis such as systemic necrotizing vasculitis, and ANCA-associated vasculitis, such as Churg-Strauss vasculitis or syndrome (CSS) and ANCA-associated small-vessel vasculitis, temporal arteritis, aplastic anemia, autoimmune aplastic anemia, Coombs positive anemia, Diamond Blackfan anemia, hemolytic anemia or immune hemolytic anemia including autoimmune hemolytic anemia (AIHA), pernicious anemia (anemia perniciosa), Addison's disease, pure red cell anemia or aplasia (PRCA), Factor VIII deficiency, hemophilia A, autoimmune neutropenia, pancytopenia, leukopenia, diseases involving leukocyte diapedesis, CNS inflammatory disorders, multiple organ injury syndrome such as those secondary to septicemia, trauma or hemorrhage, antigen-antibody complex-mediated diseases, anti-glomerular basement membrane disease, anti-phospholipid antibody syndrome, allergic neuritis, Behcet's disease/syndrome, Castleman's syndrome, Goodpasture's syndrome, Reynaud's syndrome, Sjdgren's syndrome, Stevens-Johnson syndrome, pemphigoid such as pemphigoid bullous and skin pemphigoid, pemphigus (including pemphigus vulgaris, pemphigus foliaceus, pemphigus mucus-membrane pemphigoid, and pemphigus erythematosus), autoimmune polyendocrinopathies, Reiter's disease or syndrome, thermal injury, preeclampsia, an immune complex disorder such as immune complex nephritis, antibody-mediated nephritis, polyneuropathies, chronic neuropathy such as IgM polyneuropathies or IgM-mediated neuropathy, thrombocytopenia (as developed by myocardial infarction patients, for example), including thrombotic thrombocytopenic purpura (TTP), post-transfusion purpura (PTP), heparin-induced thrombocytopenia, and autoimmune or immune-mediated thrombocytopenia such as idiopathic thrombocytopenic purpura (ITP) including chronic or acute ITP, scleritis such as idiopathic cerato-scleritis, episcleritis, autoimmune disease of the testis and ovary including autoimmune orchitis and oophoritis, primary hypothyroidism, hypoparathyroidism, autoimmune endocrine diseases including thyroiditis such as autoimmune thyroiditis, Hashimoto's disease, chronic thyroiditis (Hashimoto's thyroiditis), or subacute thyroiditis, autoimmune thyroid disease, idiopathic hypothyroidism, Grave's disease, polyglandular syndromes such as autoimmune polyglandular syndromes (or polyglandular endocrinopathy syndromes), paraneoplastic syndromes, including neurologic paraneoplastic syndromes such as Lambert-Eaton myasthenic syndrome or Eaton-Lambert syndrome, stiff-man or stiff-person syndrome, encephalomyelitis such as allergic encephalomyelitis or encephalomyelitis allergica and experimental allergic encephalomyelitis (EAE), myasthenia gravis such as thymoma-associated myasthenia gravis, cerebellar degeneration, neuromyotonia, opsoclonus or opsoclonus myoclonus syndrome (OMS), and sensory neuropathy, multifocal motor neuropathy, Sheehan's syndrome, autoimmune hepatitis, chronic hepatitis, lupoid hepatitis, giant-cell hepatitis, chronic active hepatitis or autoimmune chronic active hepatitis, lymphoid interstitial pneumonitis (LIP), bronchiolitis obliterans (non-transplant) vs NSIP, Guillain-Barré syndrome, Berger's disease (IgA nephropathy), idiopathic IgA nephropathy, linear IgA dermatosis, acute febrile neutrophilic dermatosis, subcomeal pustular dermatosis, transient acantholytic dermatosis, cirrhosis such as primary biliary cirrhosis and pneumonocirrhosis, autoimmune enteropathy syndrome, Celiac or Coeliac disease, celiac sprue (gluten enteropathy), refractory sprue, idiopathic sprue, cryoglobulinemia, amylotrophic lateral sclerosis (ALS; Lou Gehrig's disease), coronary artery disease, autoimmune ear disease such as autoimmune inner ear disease (AIED), autoimmune hearing loss, polychondritis such as refractory or relapsed or relapsing polychondritis, pulmonary alveolar proteinosis, Cogan's syndrome/nonsyphilitic interstitial keratitis, Bell's palsy, Sweet's disease/syndrome, rosacea autoimmune, zoster-associated pain, amyloidosis, a non-cancerous lymphocytosis, a primary lymphocytosis, which includes monoclonal B cell lymphocytosis (e.g., benign monoclonal gammopathy and monoclonal gammopathy of undetermined significance, MGUS), peripheral neuropathy, paraneoplastic syndrome, channelopathies such as epilepsy, migraine, arrhythmia, muscular disorders, deafness, blindness, periodic paralysis, and channelopathies of the CNS, autism, inflammatory myopathy, focal or segmental or focal segmental glomerulosclerosis (FSGS), endocrine opthalmopathy, uveoretinitis, chorioretinitis, autoimmune hepatological disorder, fibromyalgia, multiple endocrine failure, Schmidt's syndrome, adrenalitis, gastric atrophy, presenile dementia, demyelinating diseases such as autoimmune demyelinating diseases and chronic inflammatory demyelinating polyneuropathy, Dressler's syndrome, alopecia areata, alopecia totalis, CREST syndrome (calcinosis, Raynaud's phenomenon, esophageal dysmotility, sclerodactyly, and telangiectasia), male and female autoimmune infertility, e.g., due to anti-spermatozoan antibodies, mixed connective tissue disease, Chagas' disease, rheumatic fever, recurrent abortion, farmer's lung, erythema multiforme, post-cardiotomy syndrome, Cushing's syndrome, bird-fancier's lung, allergic granulomatous angiitis, benign lymphocytic angiitis, Alport's syndrome, alveolitis such as allergic alveolitis and fibrosing alveolitis, interstitial lung disease, transfusion reaction, leprosy, malaria, parasitic diseases such as leishmaniasis, kypanosomiasis, schistosomiasis, ascariasis, aspergillosis, Sampter's syndrome, Caplan's syndrome, dengue, endocarditis, endomyocardial fibrosis, diffuse interstitial pulmonary fibrosis, interstitial lung fibrosis, pulmonary fibrosis, idiopathic pulmonary fibrosis, cystic fibrosis, endophthalmitis, erythema elevatum et diutinum, erythroblastosis fetalis, eosinophilic faciitis, Shulman's syndrome, Felty's syndrome, flariasis, cyclitis such as chronic cyclitis, heterochronic cyclitis, iridocyclitis (acute or chronic), or Fuch's cyclitis, Henoch-Schonlein purpura, human immunodeficiency virus (HIV) infection, SCID, acquired immune deficiency syndrome (AIDS), echovirus infection, sepsis, endotoxemia, pancreatitis, thyroxicosis, parvovirus infection, rubella virus infection, post-vaccination syndromes, congenital rubella infection, Epstein-Barr virus infection, mumps, Evan's syndrome, autoimmune gonadal failure, Sydenham's chorea, post-streptococcal nephritis, thromboangitis ubiterans, thyrotoxicosis, tabes dorsalis, chorioiditis, giant-cell polymyalgia, chronic hypersensitivity pneumonitis, keratoconjunctivitis sicca, epidemic keratoconjunctivitis, idiopathic nephritic syndrome, minimal change nephropathy, benign familial and ischemia-reperfusion injury, transplant organ reperfusion, retinal autoimmunity, joint inflammation, bronchitis, chronic obstructive airway/pulmonary disease, silicosis, aphthae, aphthous stomatitis, arteriosclerotic disorders, aspermiogenese, autoimmune hemolysis, Boeck's disease, cryoglobulinemia, Dupuytren's contracture, endophthalmia phacoanaphylactica, enteritis allergica, erythema nodosum leprosum, idiopathic facial paralysis, chronic fatigue syndrome, febris rheumatica, Hamman-Rich's disease, sensoneural hearing loss, haemoglobinuria paroxysmatica, hypogonadism, ileitis regionalis, leucopenia, mononucleosis infectiosa, traverse myelitis, primary idiopathic myxedema, nephrosis, ophthalmia symphatica, orchitis granulomatosa, pancreatitis, polyradiculitis acuta, pyoderma gangrenosum, Quervain's thyreoiditis, acquired spenic atrophy, non-malignant thymoma, vitiligo, toxic-shock syndrome, food poisoning, conditions involving infiltration of T cells, leukocyte-adhesion deficiency, immune responses associated with acute and delayed hypersensitivity mediated by cytokines and T-lymphocytes, diseases involving leukocyte diapedesis, multiple organ injury syndrome, antigen-antibody complex-mediated diseases, antiglomerular basement membrane disease, allergic neuritis, autoimmune polyendocrinopathies, oophoritis, primary myxedema, autoimmune atrophic gastritis, sympathetic ophthalmia, rheumatic diseases, mixed connective tissue disease, nephrotic syndrome, insulitis, polyendocrine failure, autoimmune polyglandular syndrome type I, adult-onset idiopathic hypoparathyroidism (AOIH), cardiomyopathy such as dilated cardiomyopathy, epidermolisis bullosa acquisita (EBA), hemochromatosis, myocarditis, nephrotic syndrome, primary sclerosing cholangitis, purulent or nonpurulent sinusitis, acute or chronic sinusitis, ethmoid, frontal, maxillary, or sphenoid sinusitis, an eosinophil-related disorder such as eosinophilia, pulmonary infiltration eosinophilia, eosinophilia-myalgia syndrome, Loffler's syndrome, chronic eosinophilic pneumonia, tropical pulmonary eosinophilia, bronchopneumonic aspergillosis, aspergilloma, or granulomas containing eosinophils, anaphylaxis, seronegative spondyloarthritides, polyendocrine autoimmune disease, sclerosing cholangitis, sclera, episclera, chronic mucocutaneous candidiasis, Bruton's syndrome, transient hypogammaglobulinemia of infancy, Wiskott-Aldrich syndrome, ataxia telangiectasia syndrome, angiectasis, autoimmune disorders associated with collagen disease, rheumatism, neurological disease, lymphadenitis, reduction in blood pressure response, vascular dysfunction, tissue injury, cardiovascular ischemia, hyperalgesia, renal ischemia, cerebral ischemia, and disease accompanying vascularization, allergic hypersensitivity disorders, glomerulonephritides, reperfusion injury, ischemic re-perfusion disorder, reperfusion injury of myocardial or other tissues, lymphomatous tracheobronchitis, inflammatory dermatoses, dermatoses with acute inflammatory components, multiple organ failure, bullous diseases, renal cortical necrosis, acute purulent meningitis or other central nervous system inflammatory disorders, ocular and orbital inflammatory disorders, granulocyte transfusion-associated syndromes, cytokine-induced toxicity, narcolepsy, acute serious inflammation, chronic intractable inflammation, pyelitis, endarterial hyperplasia, peptic ulcer, valvulitis, and endometriosis.

### 5. MCAM Antagonists

The present invention provides antagonists of MCAM. Such antagonists encompass those that directly act upon MCAM (e.g., an anti-MCAM antibody) and those that indirectly affect MCAM activity (e.g., an anti-laminin α4 chain antibody). Such antagonists are useful, for example, for treating a central nervous system (CNS) inflammatory disorder characterized by infiltration of MCAM-expressing cells into the CNS. In one embodiment, a composition comprising an MCAM antagonist is useful for reducing inflammation in a mammalian subject. In another embodiment, such a composition is useful for partially or fully inhibiting CNS infiltration of MCAM-expressing cells. Examples of MCAM antagonists include, without limitation, antagonist or neutralizing antibodies or antibody fragments against one or more domains, e.g., an immunoglobulin domain of a native sequence MCAM polypeptide or a domain of a native sequence laminin α4 chain polypeptide (e.g., the α4 chain of laminin 411), small molecules, ribozymes, aptamers, peptides, and nucleic acids that encode polypeptide antagonists or antagonist antibodies. Reference to “an” antagonist encompasses a single antagonist. In one embodiment, the MCAM antagonists are antibodies including, without limitation, chimeric, humanized and human antibodies and their functional fragments.

In a preferred embodiment, the laminin α4 chain is an α4 chain of laminin 411. In another preferred embodiment, the MCAM antagonist blocks the interaction of an MCAM domain comprising the amino acid sequence of SEQ ID NO:22 and/or SEQ ID NO:23 with a laminin α4 chain.

### 5.1 Screening Assays to Identify MCAM Antagonists

The present invention includes screening assays to identify MCAM antagonists, which find utility in the treatment of inflammatory conditions characterized by infiltration of MCAM-expressing cells into the central nervous system (CNS).

In one aspect, the invention concerns a method for identifying an inhibitor of CNS infiltration by MCAM-expressing cells comprising the steps of: (a) incubating a population of cells expressing a laminin α4 chain, e.g., an α4 chain of laminin 411, with MCAM, in the presence or absence of a candidate molecule; (b) monitoring the level of binding of MCAM to the cells; and (c) identifying said candidate molecule as an inhibitor of CNS infiltration by MCAM-expressing cells if the level of MCAM binding is lower in the presence than in the absence of said candidate molecule. In one embodiment, the candidate molecule is selected from the group consisting of a small molecule, a peptide, a polypeptide, and an antibody. Those of ordinary skill in the art will appreciate that other types of candidate molecule may be suitable. In another embodiment, the level of binding of MCAM is monitored by known techniques including, without limitation, fluorescent microscopy, FACS, and ELISA. In one other embodiment, the cells expressing a laminin α4 chain are endothelial cells. In a preferred embodiment, the laminin α4 chain is an α4 chain of laminin 411.

Screening assays for antagonist drug candidates may be designed to identify compounds that bind or complex with MCAM (including a subunit or other fragment thereof) or with an MCAM ligand, such as a laminin α4 chain (e.g., an α4 chain of laminin 411), or otherwise interfere with the interaction of MCAM with other cellular proteins, thereby interfering with the interaction of MCAM with its ligand, e.g., a laminin α4 chain. The screening assays provided herein include assays amenable to high-throughput screening of chemical libraries, making them particularly suitable for identifying small molecule drug candidates. Generally, binding assays and activity assays are provided.

The assays can be performed in a variety of formats, including, without limitation, protein-protein binding assays, biochemical screening assays, immunoassays, and cell-based assays, which are well characterized in the art.

All assays for antagonists and agonists are common in that they call for contacting the drug candidate with an MCAM polypeptide, or an MCAM ligand polypeptide, e.g., a laminin α4 chain, or a fragment of such polypeptides (specifically including MCAM and laminin α4 chains) under conditions and for a time sufficient to allow these two components to interact.

For example, human MCAM is a 646 amino acid polypeptide, the sequence of which is available from the GenBank database under Accession Number AAA20922.1 (CAA48332) (SEQ ID NO: 11; FIG. 11A). Amino acid sequences for human laminin α4-chain are available from the GenBank database under Accession Nos. NP001098676 and NP001098677 (SEQ ID NOS:27-28; FIG. 12A-B). The making of antibodies or small molecules binding to such polypeptides is well within the skill of the ordinary artisan.

In binding assays, the interaction is binding, and the complex formed can be isolated or detected in the reaction mixture. In a particular embodiment, either the MCAM or MCAM ligand polypeptide or the drug candidate is immobilized on a solid phase, e.g., on a microtiter plate, by covalent or non-covalent attachments. Non-covalent attachment generally is accomplished by coating the solid surface with a solution of the MCAM or MCAM ligand polypeptide and drying. Alternatively, an immobilized antibody, e.g., a monoclonal antibody, specific for the MCAM or MCAM ligand polypeptide to be immobilized can be used to anchor it to a solid surface. The assay is performed by adding the non-immobilized component, which may be labeled by a detectable label, to the immobilized component, e.g., the coated surface containing the anchored component. When the reaction is complete, the non-reacted components are removed, e.g., by washing, and complexes anchored on the solid surface are detected. When the originally non-immobilized component carries a detectable label, the detection of label immobilized on the surface indicates that complexing occurred. Where the originally non-immobilized component does not carry a label, complexing can be detected, for example, by using a labeled antibody specifically binding the immobilized complex.

If the candidate compound is a polypeptide which interacts with but does not bind to MCAM or the MCAM ligand polypeptide, its interaction with the respective polypeptide can be assayed by methods well known for detecting protein-protein interactions. Such assays include traditional approaches, such as, e.g., cross-linking, co-immunoprecipitation, and co-purification through gradients or chromatographic columns. In addition, protein-protein interactions can be monitored by using a yeast-based genetic system described by Fields and co-workers (Fields and Song, Nature (London), 340:245-246 (1989); Chien et al., Proc. Natl. Acad. Sci. USA, 88:9578-9582 (1991)) as disclosed by Chevray and Nathans, Proc. Natl. Acad. Sci. USA, 89: 5789-5793 (1991).

Compounds that interfere with the interaction of MCAM and other extracellular components, in particular an MCAM ligand polypeptide, can be tested as follows. Usually a reaction mixture is prepared containing MCAM and the extracellular component (e.g., MCAM ligand such as a laminin α4 chain, e.g., an α4 chain of laminin 411) under conditions and for a time allowing for the interaction of the two products. To test the ability of a candidate compound to inhibit the interaction of MCAM and its ligand, the reaction is run in the absence and in the presence of the test compound. In addition, a placebo may be added to a third reaction mixture, to serve as positive control. Since MCAM has been shown to specifically bind its ligand, e.g., a laminin α4 chain, the ability of the test compound to inhibit the MCAM/MCAM ligand interaction can, for example, be tested by measuring the degree of binding between MCAM and its ligand in the absence and presence of the test compound. If the degree of MCAM binding to its ligand is lower in the absence of the candidate compound than in its presence, the candidate compound is an MCAM antagonist by the definition of the present invention.

An alternate screening protocol involves the use of a population of cells expressing a laminin α4 chain, e.g., an α4 chain of laminin 411, which can be incubated with MCAM, in the presence and absence of a test compound, and binding of MCAM to the cell population monitored, e.g. by fluorescent microscopy (exemplified in Example 5). Other methods of monitoring will be appreciated by those skilled in the art, including fluorescence-activated cell sorting (FACS) and enzyme-linked immunosorbent assay (ELISA). If the binding of MCAM to the cell population in the presence of the test compound is lower than in its absence, the test compound is an MCAM antagonist.

The MCAM antagonists identified based upon their ability to inhibit the binding of MCAM to its ligand, e.g., a laminin α4 chain, are drug candidates for the treatment of neruoinflammatory conditions characterized by infiltration of MCAM-expressing cells into the CNS.

It is emphasized that the screening assays specifically discussed herein are for illustration only. A variety of other assays, which can be selected depending on the type of the antagonist candidates screened (e.g. polypeptides, peptides, non-peptide small organic molecules, aptamers, ribozymes, nucleic acid, etc.) are well know to those skilled in the art and are equally suitable for the purposes of the present invention.

### 5.2 Antibodies

In one aspect, an MCAM antagonist is an anti-MCAM antibody or an anti-laminin α4 chain, e.g., α4 chain of laminin 411, antibody, or an antigen-binding fragment thereof. In some embodiments, an anti-MCAM antibody is a blocking antibody that fully or partially blocks the interaction of MCAM with its ligand, a laminin α4 chain. In other embodiments, an anti-laminin α4 chain antibody is a blocking antibody that fully or partially blocks the interaction of a laminin α4 chain with MCAM. In certain embodiments, the anti-MCAM antibody binds to the extracellular domain of MCAM which interacts with its ligand, a laminin α4 chain. In a preferred embodiment, the laminin α4 chain is an α4 chain of laminin 411.

In one embodiment, an anti-MCAM antibody specifically or selectively binds to an MCAM fragment comprising or having the amino acid sequence of position 19 to position 129 of SEQ ID NO: 11 (SEQ ID NO:22). In another embodiment, an anti-MCAM antibody specifically or selectively binds to an MCAM fragment comprising or having the amino acid sequence of position 139 to position 242 of SEQ ID NO: 11 (SEQ ID NO:23). In one other embodiment, an anti-MCAM antibody specifically or selectively binds to an MCAM fragment comprising the amino acid sequences of SEQ ID NOS:22 and 23).

In a preferred embodiment, the antagonist antibody blocks the interaction of an MCAM domain comprising the amino acid sequence of SEQ ID NO:22 and/or SEQ ID NO:23 with a laminin α4 chain.

In one other embodiment, the anti-MCAM antibody or antibody fragment comprises the following hypervariable regions (HVRs):

a) HVR-L1 shown as SEQ ID NO:3;
b) HVR-L2 shown as SEQ ID NO:4;
c) HVR-L3 shown as SEQ ID NO:5;
d) HVR-H1 shown as SEQ ID NO:8;
e) HVR-H2 shown as SEQ ID NO:9; and
f) HVR-H3 shown as SEQ ID NO:10.

In another embodiment, the anti-MCAM antibody or antibody fragment comprises a light chain variable domain shown as SEQ ID NO:2 and/or a heavy chain variable domain shown as SEQ ID NO:7. In other embodiments, the anti-MCAM antibody or antibody fragment comprises the following hypervariable regions (HVRs):

a) HVR-L1 shown as SEQ ID NO: 14;
b) HVR-L2 shown as SEQ ID NO: 15;
c) HVR-L3 shown as SEQ ID NO: 16;
d) HVR-H1 shown as SEQ ID NO: 19;
e) HVR-H2 shown as SEQ ID NO:20; and
f) HVR-H3 shown as SEQ ID NO:21.

In one other embodiment, the anti-MCAM antibody or antibody fragment comprises a light chain variable domain shown as SEQ ID NO:13 and/or a heavy chain variable domain shown as SEQ ID NO:18.

In another aspect, the present invention provides MCAM antagonists that bind to substantially the same epitope as an anti-MCAM antibody described herein. In one embodiment, the MCAM antagonist binds to substantially the same epitope as an anti-MCAM antibody comprising the following HVRs:

a) HVR-L1 shown as SEQ ID NO:3;
b) HVR-L2 shown as SEQ ID NO:4;
c) HVR-L3 shown as SEQ ID NO:5;
d) HVR-H1 shown as SEQ ID NO:8;
e) HVR-H2 shown as SEQ ID NO:9; and
f) HVR-H3 shown as SEQ ID NO:10.

In another embodiment, the MCAM antagonist binds to substantially the same epitope as an anti-MCAM antibody comprising a light chain variable domain shown as SEQ ID NO:2 and/or a heavy chain variable domain shown as SEQ ID NO:7.

In one other embodiment, the MCAM antagonist binds to substantially the same epitope as an anti-MCAM antibody comprising the following HVRs:

a) HVR-L1 shown as SEQ ID NO: 14;
b) HVR-L2 shown as SEQ ID NO: 15;
c) HVR-L3 shown as SEQ ID NO: 16;
d) HVR-H1 shown as SEQ ID NO: 19;
e) HVR-H2 shown as SEQ ID NO:20; and
f) HVR-H3 shown as SEQ ID NO:21.

In another embodiment, the MCAM antagonist binds to substantially the same epitope as an anti-MCAM antibody comprising a light chain variable domain shown as SEQ ID NO: 13 and/or a heavy chain variable domain shown as SEQ ID NO: 18.

The invention herein includes the production and use of MCAM antagonist antibodies. Exemplary methods for generating antibodies are described in more detail herein. MCAM antibodies can include, but are not limited to, polyclonal, monoclonal, multispecific, human, humanized, primatized, or chimeric antibodies, single chain antibodies (e.g., scFv), Fab fragments, F(ab′) fragments, fragments produced by a Fab expression library, anti-idiotypic (anti-Id) antibodies (including, e.g., anti-Id antibodies to antibodies of the present embodiments), and epitope-binding fragments of any of the above. Human antigen-binding antibody fragments include, but are not limited to, Fab, Fab′ and F(ab′)₂, Fd, single-chain Fvs (scFv), single-chain antibodies, disulfide-linked Fvs (sdFv), and fragments comprising either a VL or VH domain. Antigen-binding antibody fragments, including single-chain antibodies, may comprise the variable region(s) alone or in combination with the entirety or a portion of the following: hinge region, CH1, CH2, and CH3 domains. Also included are antigen-binding fragments that can comprise any combination of variable region(s) with a hinge region, CH1, CH2, and CH3 domains. The antibodies may be from any animal origin including birds and mammals. Typically, the antibodies are from human or other primates, murine (e.g., mouse and rat), donkey, sheep, monkey, rabbit, goat, guinea pig, pig, camel, horse, or chicken (or other avian). As used herein, “human” antibodies include antibodies having the amino acid sequence of a human immunoglobulin and include antibodies isolated from human immunoglobulin libraries or from animals transgenic for one or more human immunoglobulins and that do not express endogenous immunoglobulins, as described, for example in, U.S. Pat. No. 5,939,598.

In another embodiment, the MCAM antibody can be a monoclonal antibody. In yet a further embodiment, the antibody may be chemically modified, e.g., by pegylation. Additionally, other antibodies can be identified using techniques available in the art. For example, antibodies capable of specifically binding to MCAM can be produced using phage display technology. Antibody fragments that selectively bind to MCAM can then be isolated. Exemplary methods for producing such antibodies via phage display are disclosed, for example, in U.S. Pat. No. 6,225,447, for example.

Monoclonal antibodies can also be produced using the conventional hybridoma methods. These methods have been widely applied to produce hybrid cell lines that secrete high levels of monoclonal antibodies against many specific antigens, and can also be used to produce monoclonal antibodies capable of specifically binding to MCAM. For example, mice (e.g., Balb/c mice) can be immunized with an antigenic MCAM epitope by intraperitoneal injection. After sufficient time has passed to allow for an immune response, the mice are sacrificed, and the spleen cells obtained and fused with myeloma cells, using techniques well known in the art. The resulting fused cells, hybridomas, are then grown in a selective medium, and the surviving cells grown in such medium using limiting dilution conditions. After cloning and recloning, hybridomas can be isolated for secreting antibodies (for example, of the IgG or IgM class or IgG1 subclass) that selectively bind to MCAM. To produce agents specific for human use, the isolated monoclonal can then be used to produce chimeric and humanized antibodies.

MCAM antagonist antibodies are selected using an antigen derived from a mammalian species. Preferably the antigen is human MCAM or a laminin α4 chain, e.g., α4 chain of laminin 411. However, polypeptides from other species such as murine MCAM or laminin α4 chain can also be used as the target antigen. The antigens from various mammalian species may be isolated from natural sources. In other embodiments, the antigen is produced recombinantly or made using other synthetic methods known in the art. The antibody selected will normally have a sufficiently strong binding affinity for the antigen. For example, the antibody may bind human MCAM or a laminin α4 chain, e.g., an α4 chain of laminin 411 with a K_{d} value of no more than about 5 nM, preferably no more than about 2 nM, and more preferably no more than about 500 pM. Antibody affinities may be determined by a surface plasmon resonance based assay (such as the BIAcore assay as described in Examples); enzyme-linked immunoabsorbent assay (ELISA); and competition assays (e.g. RIA's), for example.

Also, the antibody may be subject to other biological activity assays, e.g., in order to evaluate its effectiveness as a therapeutic. Such assays are known in the art and depend on the target antigen and intended use for the antibody. Examples include the experimental autoimmune encephalomyelitis (EAE) (as described in Example 7 below), and in vitro and in vivo assays described herein for identifying MCAM antagonists.

To screen for antibodies which bind to a particular epitope on the antigen of interest, a routine cross-blocking assay such as that described in Antibodies, A Laboratory Manual, Cold Spring Harbor Laboratory, Ed Harlow and David Lane (1988), can be performed. Alternatively, epitope mapping, e.g. as described in Champe et al. (1995) J. Biol. Chem. 270:1388-1394, can be performed to determine whether the antibody binds an epitope of interest.

In a preferred embodiment, the antagonist antibodies are selected using a unique phage display approach. The approach involves generation of synthetic antibody phage libraries based on single framework template, design of sufficient diversities within variable domains, display of polypeptides having the diversified variable domains, selection of candidate antibodies with high affinity to target antigen, and isolation of the selected antibodies. Details of the phage display methods can be found, for example, in WO03/102157 published Dec. 11, 2003. The antibody generated from phage libraries can be further modified to generate antibody mutants with improved physical, chemical and or biological properties over the parent antibody. Where the assay used is a biological activity assay, the antibody mutant preferably has a biological activity in the assay of choice which is at least about 10 fold better, preferably at least about 20 fold better, more preferably at least about 50 fold better, and sometimes at least about 100 fold or 200 fold better, than the biological activity of the parent antibody in that assay. For example, an anti-MCAM antibody mutant preferably has a binding affinity for MCAM which is at least about 10 fold stronger, preferably at least about 20 fold stronger, more preferably at least about 50 fold stronger, and sometimes at least about 100 fold or 200 fold stronger, than the binding affinity of the parent anti-MCAM antibodies, such as clone 15 or 17 antibodies.

Chimeric and humanized antibodies can be produced from non-human antibodies, and can have the same or similar binding affinity as the antibody from which they are produced. Exemplary techniques for producing chimeric antibodies include splicing the genes from, e.g., a mouse antibody molecule of appropriate antigen specificity together with genes from a human antibody molecule of appropriate biological activity. See, e.g., Morrison et al., 1984 Proc. Nat'l. Acad. Sci. USA 81: 6851; Neuberger et al., 1984 Nature 312: 604; and Takeda et al., 1985 Nature 314: 452. For example, a nucleic acid encoding a variable (V) region of a mouse monoclonal antibody can be joined to a nucleic acid encoding a human constant (C) region, e.g., IgG1 or IgG4. The resulting antibody is thus a species hybrid, generally with the antigen binding domain from the non-human antibody and the C or effector domain from a human or primate antibody.

Humanized antibodies are antibodies with variable regions that are primarily from a human antibody (i.e., the acceptor antibody), but which have complementarity determining regions substantially from a non-human antibody (the donor antibody). See, e.g., Queen et al., Proc. Nat'l. Acad. Sci USA 86: 10029-10033 (1989); WO 90/07861, U.S. Pat. Nos. 7,435,802, 6,054,297; 5,693,761; 5,585,089; 5,530,101; and 5,224,539. The constant region or regions of these antibodies are generally also from a human antibody. The human variable domains are typically chosen from human antibodies having sequences displaying a high homology with the desired non-human variable region binding domains. The heavy and light chain variable residues can be derived from the same antibody, or a different human antibody. In addition, the sequences can be chosen as a consensus of several human antibodies, such as described in WO 92/22653.

A “Primatized™ antibody” is a recombinant antibody containing primate variable sequences or antigen binding portions, and human constant domain sequences. See e.g., Newman, Bio/Technology, 1992, 10: 1455-60. Primatization of antibodies results in the generation of antibodies that contain primate (e.g., monkey) variable domains and human constant sequences. See, e.g., U.S. Pat. No. 6,113,898. This technique modifies antibodies such that they are not rejected upon administration in humans because they are antigenic. This technique relies on immunization of cynomolgus monkeys with human antigens or receptors. This technique was developed to create high affinity monoclonal antibodies directed to human cell surface antigens.

In another aspect, specific amino acids within the human variable region can be selected for substitution based on the predicted conformation and antigen binding properties. This can be determined using techniques such as computer modeling, prediction of the behavior and binding properties of amino acids at certain locations within the variable region, and observation of effects of substitution. For example, when an amino acid differs between a non-human variable region and a human variable region, the human variable region can be altered to reflect the amino acid composition of the non-human variable region. In a specific embodiment, the antibodies used in the chronic dosage regime can be humanized antibodies as disclosed in U.S. Pat. No. 5,840,299. In another embodiment, transgenic mice containing human antibody genes can be immunized with an antigenic MCAM structure and hybridoma technology can be used to generate human antibodies that selectively bind to MCAM.

Chimeric, human and/or humanized antibodies can be produced by using recombinant expression, e.g., expression in human hybridomas (Cole et al., Monoclonal Antibodies and Cancer Therapy, Alan R. Liss, p. 77 (1985)), in myeloma cells, or in Chinese hamster ovary (CHO) cells. Alternatively, antibody coding sequences can be incorporated into transgenes for introduction into the genome of a transgenic animal and subsequent expression in the milk of the transgenic animal. See, e.g., U.S. Pat. No. 6,197,946. Exemplary suitable transgenes include, but are not limited to, transgenes having a promoter and/or enhancer from a mammary gland specific gene, for example casein or β-lactoglobulin.

### 5.3 Antibody Variants

In addition to the MCAM antagonist antibodies described herein, it is contemplated that variants of such antibodies can be prepared. Anti-MCAM antagonist antibody variants can be prepared by introducing appropriate nucleotide changes into the encoding DNA, and/or by synthesis of the desired antibody. Those skilled in the art will appreciate that amino acid changes may alter post-translational processes of the anti-MCAM antibody, such as changing the number or position of glycosylation sites.

Variations in the MCAM antagonist antibodies described herein, can be made, for example, using any of the techniques and guidelines for conservative and non-conservative mutations set forth, for instance, in U.S. Pat. No. 5,364,934. Variations may be a substitution, deletion or insertion of one or more codons encoding the antibody that results in a change in the amino acid sequence as compared with the native sequence antibody. Optionally the variation is by substitution of at least one amino acid with any other amino acid in one or more of the domains of the MCAM antagonist antibody. Guidance in determining which amino acid residue may be inserted, substituted or deleted without adversely affecting the desired activity may be found by comparing the sequence of the MCAM antagonist antibody with that of homologous known protein molecules and minimizing the number of amino acid sequence changes made in regions of high homology. Amino acid substitutions can be the result of replacing one amino acid with another amino acid having similar structural and/or chemical properties, such as the replacement of a leucine with a serine, i.e., conservative amino acid replacements. Insertions or deletions may optionally be in the range of about 1 to 5 amino acids. The variation allowed may be determined by systematically making insertions, deletions or substitutions of amino acids in the sequence and testing the resulting variants for activity exhibited by the full-length or mature native sequence.

MCAM antagonist antibody fragments are provided herein. Such fragments may be truncated at the N-terminus or C-terminus, or may lack internal residues, for example, when compared with a full length native antibody. Certain fragments lack amino acid residues that are not essential for a desired biological activity of the MCAM antagonist antibody.

MCAM antagonist antibody fragments may be prepared by any of a number of conventional techniques. Desired peptide fragments may be chemically synthesized. An alternative approach involves generating antibody or polypeptide fragments by enzymatic digestion, e.g., by treating the protein with an enzyme known to cleave proteins at sites defined by particular amino acid residues, or by digesting the DNA with suitable restriction enzymes and isolating the desired fragment. Yet another suitable technique involves isolating and amplifying a DNA fragment encoding a desired antibody or polypeptide fragment, by polymerase chain reaction (PCR). Oligonucleotides that define the desired termini of the DNA fragment are employed at the 5′ and 3′ primers in the PCR. Preferably, anti-MCAM antagonist antibody fragments share at least one biological and/or immunological activity with a native MCAM antagonist antibody disclosed herein.

In particular embodiments, conservative substitutions of interest are shown in Table 1 below under the heading of preferred substitutions. If such substitutions result in a change in biological activity, then more substantial changes, as further described below in reference to amino acid classes, are introduced and the products screened.

Substantial modifications in function or immunological identity of the MCAM antagonist antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:

(1) hydrophobic: norleucine, met, ala, val, leu, ile;

(2) neutral hydrophilic: cys, ser, thr;

(3) acidic: asp, glu;

(4) basic: asn, gln, his, lys, arg;

(5) residues that influence chain orientation: gly, pro; and

(6) aromatic: trp, tyr, phe.

Non-conservative substitutions will entail exchanging a member of one of these classes for another class. Such substituted residues also may be introduced into the conservative substitution sites or, more preferably, into the remaining (non-conserved) sites.

TABLE 1

| Original | Exemplary | Preferred |
|---|---|---|
| Residue | Substitutions | Substitutions |
| Ala (A) | val; leu; ile | val |
| Arg (R) | lys; gln; asn | lys |
| Asn (N) | gln; his; lys; arg | gln |
| Asp (D) | glu | glu |
| Cys (C) | ser | ser |
| Gln (Q) | asn | asn |
| Glu (E) | asp | asp |
| Gly (G) | pro; ala | ala |
| His (H) | asn; gln; lys; arg | arg |
| Ile (I) | leu; val; met; ala; phe; | |
| | norleucine | leu |
| Leu (L) | norleucine; ile; val; | |
| | met; ala; phe | ile |
| Lys (K) | arg; gln; asn | arg |
| Met (M) | leu; phe; ile | leu |
| Phe (F) | leu; val; ile; ala; tyr | leu |
| Pro (P) | ala | ala |
| Ser (S) | thr | thr |
| Thr (T) | ser | ser |
| Trp (W) | tyr; phe | tyr |
| Tyr (Y) | trp; phe; thr; ser | phe |
| Val (V) | ile; leu; met; phe; | |
| | ala; norleucine | leu |

The variations can be made using methods known in the art such as oligonucleotide-mediated (site-directed) mutagenesis, alanine scanning, and PCR mutagenesis. Site-directed mutagenesis [Carter et al., Nucl. Acids Res., 13:4331 (1986); Zoller et al., Nucl. Acids Res., 10:6487 (1987)], cassette mutagenesis [Wells et al., Gene, 34:315 (1985)], restriction selection mutagenesis [Wells et al., Philos. Trans. R. Soc. London SerA, 317:415 (1986)] or other known techniques can be performed on the cloned DNA to produce the MCAM antagonist antibody variant DNA.

Scanning amino acid analysis can also be employed to identify one or more amino acids along a contiguous sequence. Among the preferred scanning amino acids are relatively small, neutral amino acids. Such amino acids include alanine, glycine, serine, and cysteine. Alanine is typically a preferred scanning amino acid among this group because it eliminates the side-chain beyond the beta-carbon and is less likely to alter the main-chain conformation of the variant [Cunningham and Wells, Science, 244:1081-1085 (1989)]. Alanine is also typically preferred because it is the most common amino acid. Further, it is frequently found in both buried and exposed positions [Creighton, The Proteins, (W.H. Freeman & Co., N.Y.); Chothia, J. Mol. Biol., 150:1 (1976)]. If alanine substitution does not yield adequate amounts of variant, an isoteric amino acid can be used.

Any cysteine residue not involved in maintaining the proper conformation of the MCAM antagonist antibody also may be substituted, generally with serine, to improve the oxidative stability of the molecule and prevent aberrant crosslinking. Conversely, cysteine bond(s) may be added to the MCAM antagonist antibody to improve its stability (particularly where the antibody is an antibody fragment such as an Fv fragment).

A particularly preferred type of substitutional variant involves substituting one or more hypervariable region residues of a parent antibody (e.g., a humanized or human antibody). Generally, the resulting variant(s) selected for further development will have improved biological properties relative to the parent antibody from which they are generated. A convenient way for generating such substitutional variants involves affinity maturation using phage display. Briefly, several hypervariable region sites (e.g., 6-7 sites) are mutated to generate all possible amino substitutions at each site. The antibody variants thus generated are displayed in a monovalent fashion from filamentous phage particles as fusions to the gene III product of M13 packaged within each particle. The phage-displayed variants are then screened for their biological activity (e.g., binding affinity) as herein disclosed. In order to identify candidate hypervariable region sites for modification, alanine scanning mutagenesis can be performed to identify hypervariable region residues contributing significantly to antigen binding. Alternatively, or additionally, it may be beneficial to analyze a crystal structure of the antigen-antibody complex to identify contact points between the antibody and human MCAM or laminin 411 polypeptide. Such contact residues and neighboring residues are candidates for substitution according to the techniques elaborated herein. Once such variants are generated, the panel of variants is subjected to screening as described herein and antibodies with superior properties in one or more relevant assays may be selected for further development.

Preferred affinity matured antibodies have an affinity which is five times, more preferably 10 times, even more preferably 20 or 30 times greater than the starting antibody (generally murine, humanized or human) from which the matured antibody is prepared.

Nucleic acid molecules encoding amino acid sequence variants of the MCAM antagonist antibody are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the MCAM antagonist antibody.

Also included in the invention are antibodies that bind to the same epitope as the antibodies described herein. For example, antibodies of the invention specifically bind to an epitope that includes one or more amino acid residues on human MCAM (Accession No. AAA20922.1/CAA48332). In some embodiments, antibodies of the invention specifically bind MCAM, wherein the antibody binds to an epitope on human MCAM (e.g., Accession No. AAA20922.1/CAA48332).

Those skilled in the art will recognize that it is possible to determine, without undue experimentation, if a monoclonal antibody (e.g., fully human monoclonal antibody) has the same specificity as a monoclonal antibody of the invention (e.g., clones 15 and 17) by ascertaining whether the former prevents the latter from binding to MCAM. If the monoclonal antibody being tested competes with the monoclonal antibody of the invention, as shown by a decrease in binding by the monoclonal antibody of the invention, then the two monoclonal antibodies bind to the same, or a closely related, epitope.

An alternative method for determining whether a monoclonal antibody has the specificity of monoclonal antibody of the invention is to pre-incubate the monoclonal antibody of the invention with MCAM (e.g., an MCAM-Fc molecule exemplified in the Examples) and then add the monoclonal antibody being tested to determine if the monoclonal antibody being tested is inhibited in its ability to bind MCAM. If the monoclonal antibody being tested is inhibited then, in all likelihood, it has the same, or functionally equivalent, epitopic specificity as the monoclonal antibody of the invention.

Where antibody fragments are used, the smallest inhibitory fragment that specifically binds to the binding domain of the target protein is preferred. For example, based upon the variable-region sequences of an antibody, peptide molecules can be designed that retain the ability to bind the target protein sequence. Such peptides can be synthesized chemically and/or produced by recombinant DNA technology. See, e.g., Marasco et al., Proc. Natl. Acad. Sci. USA, 90: 7889-7893 (1993).

### 6. Methods of Use

The present invention provides MCAM antagonists as therapeutic agents for neuroinflammatory conditions, and autoimmune diseases. For the prevention, treatment or reduction in the severity of a given disease or condition, the appropriate dosage of a compound of the invention will depend on the type of disease or condition to be treated, as defined above, the severity and course of the disease or condition, whether the agent is administered for preventive or therapeutic purposes, previous therapy, the patient's clinical history and response to the compound, and the discretion of the attending physician. The compound is suitably administered to the patient at one time or over a series of treatments. Preferably, it is desirable to determine the dose-response curve and the pharmaceutical composition of the invention first in vitro, and then in useful animal models prior to testing in humans.

In one aspect, the present invention provides a method for inhibiting or blocking the interaction of MCAM expressed on T cells and laminin α4 chain, e.g., an α4 chain of laminin 411, comprising treating the T cells with an MCAM antagonist (as described herein), thereby inhibiting the interaction of MCAM with laminin α4 chain. In one embodiment, the laminin α4 chain is expressed on the surface of a cell, e.g., an endothelial cell. In a preferred embodiment, the MCAM antagonist is an anti-MCAM antibody. In another embodiment, the T cells are TH17 cells. In one other embodiment, the treatment with an MCAM antagonist is performed in vivo. In yet another embodiment, the treatment is performed in a mammalian subject. In one embodiment, the mammalian subject is a human.

In another aspect, the present invention provides a method for inhibiting or preventing extravasation of MCAM-expressing T cells into the central nervous system (CNS) comprising treating the T cells with an MCAM antagonist (as described herein), thereby inhibiting or preventing the extravasation of MCAM-expressing T cells into the CNS. In one embodiment, the MCAM antagonist blocks the interaction of MCAM with laminin α4 chain, e.g., an α4 chain of laminin 411. In a preferred embodiment, the MCAM antagonist is an anti-MCAM antibody. In one other embodiment, the laminin α4 chain is expressed on the surface of a cell, e.g., an endothelial cell. In another embodiment, the T cells are TH17 cells. In one other embodiment, the treatment with an MCAM antagonist is performed in vivo. In yet another embodiment, the treatment is performed in a mammalian subject. In one embodiment, the mammalian subject is a human.

In one other aspect, the present invention provides methods of treatment for a neuroinflammatory condition or an autoimmune disease. In one embodiment, the method comprises administering to a mammalian subject in need a therapeutically effective amount of an MCAM antagonist. In another aspect, the invention provides a method for the delaying or slowing down of the progression of a neuroinflammatory condition or an autoimmune disease. In one embodiment, the method comprises administering to subject diagnosed with the condition or disease, an effective amount of an MCAM antagonist. In another aspect, the invention provides a method for preventing indicia of a neuroinflammatory condition or an autoimmune disease. In one embodiment, the method comprises administering an effective amount of an MCAM antagonist to a subject at risk of the condition or disease, wherein the MCAM antagonist is effective against the development of indicia of the condition or disease.

### 6.1 Neuroinflammatory Conditions

In one aspect, the MCAM antagonists provide a preventative or prophylactic effect against the development of, or the progression of, clinical and/or histological and/or biochemical and/or pathological indicia (including both symptoms and signs) of neuroinflammatory conditions in a subject. In one embodiment, the neuroinflammatory condition is characterized by CNS inflammation and/or cell/tissue damage. In one embodiment, the indicia include increased glial activation, increased pro-inflammatory cytokine/chemokine levels (e.g., TNFα, INFγ, IL-1β), increased blood-brain-barrier permeability, and/or increased immune cell (e.g., leukocyte) recruitment/invasion to the CNS. In another embodiment, the neuroinflammation is progressive or chronic neuroinflammation associated with chronic activation of cells of the immune system (i.e., autoimmune-associated neuroinflammation). Chronic neuroinflammation conditions include, without limitation, relapsing multiple sclerosis (MS), chronic progressive MS, inactive MS, and Parkinson's disease (PD). In another embodiment, the subject is at risk for a neuroinflammatory condition. In general, a subject at risk will previously have had a neuroinflammatory condition as described herein, or will have a genetic predisposition for neuroinflammatory condition.

The efficacy of the treatment of neuroinflammatory conditions can be measured by various assessments commonly used in evaluating neuroinflammatory condition. For example, CNS health can be evaluated by testing for MS symptoms including, but not limited to, impaired vision (e.g., blurred or double vision, red-green color distortion, or blindness); muscle weakness in the extremities; impaired coordination and balance; partial or complete paralysis, paresthesias, transitory abnormal sensory feelings (e.g., numbness, prickling, or “pins and needles” sensations); pain; speech impediments; tremors; dizziness; hearing loss; cognitive impairments (e.g., difficulties with concentration, attention, memory, and poor judgment); and depression. MS testing may also include a lumbar puncture (spinal tap) for cerebrospinal fluid (CSF) tests (e.g., CSF oligoclonal banding suggesting inflammation of the CNS); a magnetic resonance imaging (MRI) scan of the head or spine; and a nerve function test (e.g., evoked potential test).

CNS health may also be evaluated by testing for PD symptoms including, but not limited to, tremor (e.g., trembling in hands, arms, legs, jaw, and face); rigidity or stiffness of the limbs and trunk; bradykinesia or slowness of movement; postural instability or impaired balance and coordination; depression and other emotional changes; difficulty in swallowing, chewing, and speaking; urinary problems or constipation; skin problems; sleep disruptions; and brain scans or other tests to rule out other diseases.

### 6.2 Autoimmune Diseases

For autoimmune diseases, the term “treatment” refers to both therapeutic treatment and prophylactic or preventative measures for an autoimmune disease, wherein the object is to prevent or slow down (lessen) the targeted pathologic condition or disorder. Those in need of treatment include those already with an autoimmune disease as well as those prone to have an autoimmune disease or those in whom the autoimmune disease is to be prevented.

In one aspect, the MCAM antagonists provide a preventative or prophylactic effect against the development of, or the progression of, clinical and/or histological and/or biochemical and/or pathological indicia (including both symptoms and signs) of autoimmune disease in a subject. In another embodiment, the subject is at risk for autoimmune disease or an autoimmune disease flare-up. In general, a subject at risk will previously have had autoimmune disease and/or one or more autoimmune disease flare-ups, or will have a genetic predisposition for an autoimmune disease.

In one embodiment, the present invention provides an MCAM antagonist for use as a medicament for, or for the treatment of a disease, condition or disorder described herein. In another embodiment, the present invention provides the use of an MCAM antagonist for the manufacture of a medicament for treating a disease, condition or disorder described herein. In one other embodiment, the present invention provides the use of an MCAM antagonist described herein, in the manufacture of a medicament for the treatment of a central nervous system (CNS) inflammatory disorder characterized by infiltration of MCAM-expressing cells into the CNS.

### 7. Pharmaceutical Compositions

MCAM antagonist antibodies specifically binding MCAM or a laminin α4 chain, e.g., an α4 chain of laminin 411, as well as other MCAM antagonist molecules identified by the screening assays disclosed hereinbefore, can be administered for the treatment of various disorders, in particular neuroinflammatory diseases or diseases benefiting from the inhibition of the infiltration of MCAM-expressing cells into the CNS, in the form of pharmaceutical compositions.

In one aspect, the present invention concerns pharmaceutical compositions comprising an antibody, or antigen binding fragment thereof, as described herein. In one embodiment, the pharmaceutical composition comprises

(i) an isolated anti-MCAM antibody, or antigen binding fragment thereof, that binds to an immunoglobulin domain of MCAM comprising the amino acid sequence shown as SEQ ID NO:22;

(ii) an isolated anti-MCAM antibody, or antigen binding fragment thereof, that binds to an immunoglobulin domain of MCAM comprising the amino acid sequence shown as SEQ ID NO:23; or

(iii) an isolated anti-MCAM antibody, or antigen binding fragment thereof, that binds to a domain of MCAM comprising the amino acid sequences shown as SEQ ID NOS:22 and 23.

In another embodiment, the pharmaceutical composition comprises an isolated anti-MCAM antibody, or antigen binding fragment thereof, comprising the following hypervariable regions (HVRs):

(i) an HVR-L1 comprising the amino acid sequence KASKNIDTYLA (SEQ ID NO:3);

(ii) an HVR-L2 comprising the amino acid sequence SGSTL (SEQ ID NO:4);

(iii) an HVR-L3 comprising the amino acid sequence QQHNEYPLT (SEQ ID NO:5);

(iv) an HVR-H1 comprising the amino acid sequence GFTFSNYYMA (SEQ ID NO: 8)

(v) an HVR-H2 comprising the amino acid sequence SISFEGNRNHYGDSVK (SEQ ID NO:9); and

(vi) an HVR-H3 comprising the amino acid sequence HRGYSTNFYHDVLDAWGQG (SEQ ID NO: 10).

In one other embodiment, the pharmaceutical composition comprises an isolated anti-MCAM antibody, or antigen binding fragment thereof, comprising the following hypervariable regions (HVRs):

(i) an HVR-L1 comprising the amino acid sequence KSSQSLLYSGTQKNYLA (SEQ ID NO: 14);

(ii) an HVR-L2 comprising the amino acid sequence WASTRQS (SEQ ID NO: 15);

(iii) an HVR-L3 comprising the amino acid sequence QQYYDTLTDT (SEQ ID NO:16);

(iv) an HVR-H1 comprising the amino acid sequence GFKFSNYYMS (SEQ ID NO:19);

(v) an HVR-H2 comprising the amino acid sequence SISDGGGDTFCRDLVKG (SEQ ID NO:20); and

(vi) an HVR-H3 comprising the amino acid sequence RGAAMGGVMDAWGQG (SEQ ID NO:21).

In another embodiment, the pharmaceutical composition comprises an isolated anti-MCAM antibody, or antigen binding fragment thereof, comprising

(a) a light chain variable domain comprising the amino acid sequence shown as SEQ ID NO:2 and a heavy chain variable domain comprising the amino acid sequence shown as SEQ ID NO:7; or

(b) a light chain variable domain comprising the amino acid sequence shown as SEQ ID NO: 13 and a heavy chain variable domain comprising the amino acid sequence shown as SEQ ID NO:18.

In yet another embodiment, the pharmaceutical composition comprises an isolated anti-MCAM antibody, or antigen binding fragment thereof, which binds to substantially the same epitope as an antibody described herein. In one other embodiment, the pharmaceutical composition comprises an isolated anti-MCAM antibody, or antigen binding fragment thereof, that competes for binding to human MCAM with an antibody described herein. In additional embodiments, the present invention provides the use of an anti-MCAM antibody, or antigen binding fragment thereof, as described herein, in the manufacture of a medicament for the treatment of a central nervous system (CNS) inflammatory disorder characterized by infiltration of MCAM-expressing cells into the CNS.

The compounds of the invention for prevention or treatment of a neuroinflammatory condition or autoimmune disease are typically administered by intravenous injection. Other methods administration by also be used, which includes but is not limited to, topical, parenteral, subcutaneous, intraperitoneal, intrapulmonary, intranasal, ocular, intraocular, intravitreal, intralesional, intracerobrospinal, intra-articular, intrasynovial, intrathecal, oral, topical, or inhalation administration. Parenteral infusions include intramuscular, intravenous, intraarterial, intraperitoneal, or subcutaneous administration. In addition, the compounds described herein are administered to a human subject, in accord with known methods, such as intravenous administration as a bolus or by continuous infusion over a period of time.

The present invention provides dosages for the MCAM antagonist-based therapeutics. For example, depending on the type and severity of the disease, about 1 μg/kg to 15 mg/kg (e.g. 0.1-20 mg/kg) of polypeptide is an initial candidate dosage for administration to the patient, whether, for example, by one or more separate administrations, or by continuous infusion. A typical daily dosage might range from about 1 μg/kg to 100 mg/kg or more, depending on the factors mentioned above. For repeated administrations over several days or longer, depending on the condition, the treatment is sustained until a desired suppression of disease symptoms occurs. However, other dosage regimens may be useful. The progress of this therapy is easily monitored by conventional techniques and assays.

The MCAM antagonist (including MCAM antagonist antibody) compositions herein will be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “therapeutically effective amount” of the antagonist to be administered will be governed by such considerations, and is the minimum amount necessary to prevent, ameliorate, or treat a given disease or condition.

In some embodiments, the composition is used to prevent the occurrence or reoccurrence of the disease or condition disease in the subject. In one embodiment, the present invention can be used for increasing the duration of survival of a human patient susceptible to or diagnosed with the disease or condition disease. Duration of survival is defined as the time from first administration of the drug to death.

Therapeutic formulations are prepared using standard methods known in the art by mixing the active ingredient having the desired degree of purity with optional physiologically acceptable carriers, excipients or stabilizers (see, e.g., Alfonso R Gennaro (ed), Remington: The Science and Practice of Pharmacy, formerly Remington's Pharmaceutical Sciences 20th ed., Lippincott, Williams & Wilkins, 2003, incorporated herein by reference in its entirety). Acceptable carriers, include saline, or buffers such as phosphate, citrate and other organic acids; antioxidants including ascorbic acid; low molecular weight (less than about 10 residues) polypeptides; proteins, such as serum albumin, gelatin or immunoglobulins; hydrophilic polymers such as polyvinylpyrrolidone, amino acids such as glycine, glutamine, asparagines, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, PLURONICS™, or PEG.

Optionally, but preferably, the formulation contains a pharmaceutically acceptable salt, preferably sodium chloride, and preferably at about physiological concentrations.

Optionally, the formulations of the invention can contain a pharmaceutically acceptable preservative. In some embodiments the preservative concentration ranges from 0.1 to 2.0%, typically v/v. Suitable preservatives include those known in the pharmaceutical arts. Benzyl alcohol, phenol, m-cresol, methylparaben, and propylparaben are preferred preservatives. Optionally, the formulations of the invention can include a pharmaceutically acceptable surfactant at a concentration of 0.005 to 0.02%.

The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences, supra.

Sustained-release preparations may be prepared. Suitable examples of sustained-release preparations include semipermeable matrices of solid hydrophobic polymers containing the antibody, which matrices are in the form of shaped articles, e.g., films, or microcapsule. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and .gamma. ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

### 8. Articles of Manufacture and Kits

The instant invention further includes kits comprising the MCAM antagonists of the invention and related materials, such as instructions for use. The instructions for use may contain, for example, instructions for administration of the MCAM antagonists and optionally one or more additional agents. The invention also provides kits for the treatment of a central nervous system (CNS) inflammatory disorder characterized by infiltration of MCAM-expressing cells into the CNS. The disorders include, without limitation, neuroinflammatory conditions, such as, for example, multiple sclerosis and Parkinson's disease, and autoimmune disease. The kits of the invention comprise one or more containers of at least one MCAM antagonist, preferably an antibody, in combination with a set of instructions, generally written instructions, relating to the use and dosage of the MCAM antagonist for the treatment of the disorder. The instructions included with the kit generally include information as to dosage, dosing schedule, and route of administration for the treatment of the target disorder, such as a neuroinflammatory condition or an autoimmune disease. The containers of MCAM antagonist(s) may be unit doses, bulk packages (e.g., multi-dose packages), or sub-unit doses.

In one aspect, the present invention provides a kit comprising an MCAM antagonist as described herein and instructions for use in the treatment of a central nervous system (CNS) inflammatory disorder characterized by infiltration of MCAM-expressing cells into the CNS. In one embodiment, the present invention provides a kit for the treatment of a central nervous system (CNS) inflammatory disorder characterized by infiltration of MCAM-expressing cells into the CNS, said kit comprising: (a) a container comprising an MCAM antagonist antibody; and (b) a label or instructions for administering said antibody to treat said CNS inflammatory disorder. Preferably, the CNS inflammatory disorder is a neuroinflammatory condition or an autoimmune disease. In one embodiment, the CNS inflammatory disorder is multiple sclerosis or Parkinson's disease.

Also provided is an article of manufacture for therapeutic use, comprising a container and a label or package insert on or associated with the container. Suitable containers include, for example, bottles, vials, syringes, etc. The containers may be formed from a variety of materials such as glass or plastic. The container holds a composition which is effective for treating the condition and may have a sterile access port (for example the container may be an intravenous solution bag or a vial having a stopper pierceable by a hypodermic injection needle). At least one active agent in the composition is an MCAM antagonist of the invention. The label or package insert indicates that the composition is used for treating the particular condition. The label or package insert will further comprise instructions for administering the antibody composition to the patient. Articles of manufacture and kits comprising combinatorial therapies described herein are also contemplated.

Package insert refers to instructions customarily included in commercial packages of therapeutic products that contain information about the indications, usage, dosage, administration, contraindications and/or warnings concerning the use of such therapeutic products

Additionally, the article of manufacture may further comprise a second container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### EXAMPLES

The following examples are not to be interpreted as limiting, but are exemplary means of using the methods disclosed.

Materials and Methods

Animals and Manipulation of Cells

SJL mice (Jackson), 8-16 week old, were immunized with PLP 139-151 peptide emulsified in CFA. The commercial kit, EK-0122 (Hooke Laboratories) was used for this immunization experiment. For some experiments, spleens were removed 11 days later and processed into a single cell suspension. For some experiments, splenocytes were processed for in vitro analysis as described below. For EAE studies, mice were injected on days 5, 9, 13, and 17 after PLP immunization with either PBS, isotype control antibody (BioXcell), or anti-MCAM clone 17. Progression of the disease was monitored daily and scored in a blinded fashion by standard techniques. Mice were sacrificed 35 days after PLP immunization, and brains and spinal cords were analyzed for infiltration of immune cells.

For analysis of MCAM-Fc binding to EAE tissues, 8-16 week old C57BL6 mice were immunized with myelin oligodendrocyte glycoprotein (MOG) 35-55 emulsified in CFA. The commercial kit, EK-0111 (Hooke laboratories) was used for this immunization experiment. The immunized animals were sacrificed at the peak of disease. Brains and spinal cords were snap frozen in OCT (optimal cutting temperature media) and analyzed by fluorescent microscopy as described below.

Flow Cytometry/Marker Staining and Detection/FACS Protocols

Buffy coats were obtained from healthy human donors (Stanford Blood Center, Palo Alto, Calif.) and CD4 T cells were negatively enriched using RosetteSep (Stem Cell Technologies). Where indicated, CD4+/CD45RO+ memory T cells were further negatively purified using magnetic beads (Miltenyi Biotec). T cells were plated (2×10⁵ cells/well) in anti-CD3 (5 μg/ml, BD Pharmingen) coated 96 well U bottom plates in RPMI containing 10% heat-inactivated FCS (HyClone Laboratories), penicillin, streptomycin, L-glutamine, anti-IFNγ (5 μg/ml; R&D Systems), anti-IL4 (0.5 μg/ml, R&D Systems), and anti-CD28 (2 μg/ml; BD Pharmingen) for five days. Where indicated, TGFβ (2 ng/ml, unless otherwise indicated), IL12, IL1β, and/or IL-23 (all at 20 ng/ml) were added. All cytokines were obtained from R&D Systems. Analysis of intracellular cytokines occurred following five hours in the presence of PMA (50 ng/ml) and Ionomycin (500 ng/ml; both from Sigma-Aldrich) and GolgiStop (BD Pharmingen). Surface staining with anti-MCAM (Pharmingen) was followed by fixation, permeabilization, and staining with anti-IL-17A (Ebioscience), IL-22 (R&D Systems), CCL20 (R&D Systems) and/or FOXP3 using a FOXP3 staining kit (Biolegend). In some experiments, unmanipulated whole blood was stained for surface expression with anti-CCR7, anti-CCR6, anti-Integrin alpha 4, anti-integrin beta 7, or anti-integrin beta 1 (all from BD Pharmingen).

Antibody Generation/Characterization

MCAM-Fc was generated by fusing the extracellular domain of murine MCAM to human IgG and produced in CHO cells using standard techniques. Lou/M rats were immunized with 100 μg of MCAM-Fc protein in CFA (1:1 volume). Rats were boosted two times at two week intervals with MCAM-Fc protein in incomplete Freund's adjuvant (IFA) (1:1 volume). Hybridomas were generated from immunized rats using standard protocols and clones were selected by Clonepix. CHO cells were transfected with the full length murine MCAM gene and selected for stable expression using neomycin and standard techniques. Parental CHO cells (MCAM negative) were fluorescently labeled with carboxyfluorescein succinimidyl ester (CFSE) using standard techniques and mixed at a 1:1 ratio with unlabeled MCAM transfected CHO cells. Hybridoma supernatants were incubated with this mixture of cells for 30 minutes and binding of potential MCAM specific antibodies was detected with a fluorescently labeled anti-rat secondary antibody (Jackson Immuno) by flow cytometry.

Supematants from hybridomas that screened positive for MCAM specific antibodies were pre-incubated with fluorescently labeled mouse MCAM-Fc protein (5 μg/mL) for 30 minutes before addition to the laminin α4 expressing cell line WM2664 and neutralization of binding of the MCAM-Fc protein to the cell line was determined by flow cytometry.

Nucleic Acid and Protein Manipulation

For microarray experiments, human CD4+ T cells were isolated as above, stained for CD161 and CCR6 (both from BD Pharmingen), and sorted into CD4+/CD161−/CCR6−(non-TH17) and CD4+/CD161+/CCR6+(TH17) cells from three independent healthy donors. RNA was isolated from half of the cells from each donor immediately (circulating) and the other half was stimulated with plate bound anti-CD3 and soluble anti-CD28 as above, in the absence of exogenous cytokines for four days (activated) before RNA isolation. RNA was amplified (Nugen) and hybridized on Human U133 Plus 2.0 Array (Affymetrix). All microarray experiments were performed at Expression Analysis, Inc. (Durham, N.C.).

For determination of CDRs, total RNA was isolated from hybridoma cells using RNAquous-4PCR kit (Ambion), and was used for cDNA synthesis. First and second strand cDNA was synthesized using methods modified from Marathon cDNA amplification (Clontech) with the cDNA adaptor ligated to the 5′-end of the obtained dscDNA. The reverse specific primer was designed based on the specific antibody isotype constant region sequence for both heavy and light chains, and was used along with the adaptor primer in the PCR amplification of both VL and VH fragments using Pfu Ultra DNA polymerase (Stratagene). The amplified PCR product was cloned into pCR-Blunt-TOPO (Invitrogen), and the nucleotide sequence was determined. Identical VL and VH sequences (those of clone 17) were identified from at least 3 out of 5 individual clones for both light and heavy chains.

For determination of IL-17 concentrations in the supernatant, ELISA was performed using a commercial kit (R&D Systems).

Fluorescence Microscopy/Standard Immunofluorescent Methods

Tissues from EAE induced mice were snap frozen in OCT and sectioned at 10 μM. Sections were fixed in cold acetone and stained with directly conjugated anti-pan-laminin (Novus Biologicals), MCAM-Fc, anti-CD31 (BD Pharmingen), or anti-laminin α4 (Novus biological). In some experiments, MCAM-Fc was preincubated with anti-MCAM antibodies prior to addition to tissues to ascertain neutralization of MCAM binding to its ligand on tissues.

Mouse Polarization Experiment

Splenocytes from mice immunized with PLP in CFA for 11 days were isolated and cultured in the presence of PLP (5 μg/mL, Hooke Laboratories). Where indicated, human TGFβ (5 ng/ml) and/or murine IL-23 (20 ng/mL), and murine IL-1β (20 ng/mL) were added for five days in RPMI containing 10% heat-inactivated FCS (HyClone Laboratories), penicillin, streptomycin, L-glutamine, anti-IFNγ (5 μg/ml; R&D Systems), anti-IL4 (0.5 μg/ml, R&D Systems) and (β-ME (50 μM). All cytokines were from R&D Systems. Cells were stained with anti-CD4, anti-NK1.1 (both from BD Pharmingen) and anti-MCAM generated as described above.

### Example 1. MCAM is a Gene Up-Regulated in IL-17-Producing Human CD4+ T Cells

To identify novel targetable molecules associated with TH17 cell infiltration of the CNS, human CD4+ T cells from three healthy donors were enriched by magnetic negative selection as described in Materials and Methods above. After the enriched human CD4+ T cells were stained for surface expression of CD161 and CCR6, cells were FACS sorted into two populations: CCR6−/CD161−(representing circulating non-TH17 cells) and CCR6+/CD161+(representing circulating TH17 cells) as described in Materials and Methods above. RNA was isolated immediately from half of the cells in each population as described in Materials and Methods above. The other half was put into culture with plate-bound anti-CD3 and soluble anti-CD28, without exogenous cytokines, for four days to obtain activated non-TH17 cells and activated TH17 cells, respectively. RNA was similarly isolated from these two types of activated cells. RNA was subject to microarray analysis as described in Materials and Methods above to identify genes specifically expressed in TH17 cells.

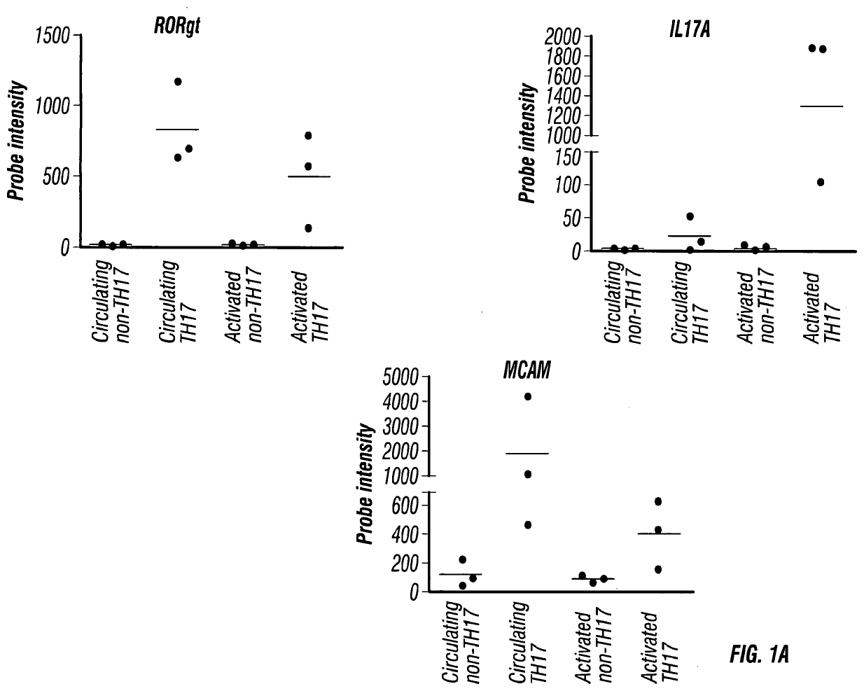

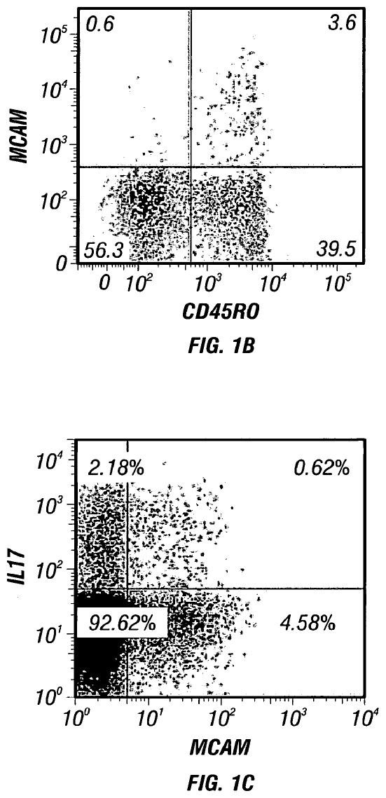

As shown in FIG. 1A, RORγt, a known TH17 transcription factor, was up-regulated in both circulating and activated TH17 cells, while IL-17, as an activated TH17 marker, was nearly exclusively expressed in the activated TH17 population. These results indicate that the above procedures of separation and activation were successful. Microarray analysis identified MCAM as an up-regulated gene in both circulating and activated TH17 cells—a profile similar to that of RORγt (FIG. 1A).

MCAM expressing T cells have been described previously as having enriched expression among T cell clones generated from multiple sclerosis patients, and are particularly prominent at sites of inflammation. See, e.g., Brucklacher-Waldert et al., Brian 132: 3329-3341 (2009); see also Pickl et al., J. Immunol. 158: 2107-2115 (1997). Here, the MCAM protein was found to be present on the surface of a small population of CD4+ T cells (typically 3-5% of healthy donors). MCAM protein was also found to exist nearly entirely with in the CD45RO+ memory population of T cells (FIG. 1B). The human CD4+ T cells were isolated as above, and stimulated for four hours with phorbol myristate acetate (PMA)/Ionomycin. The stimulated CD4+ T cells were analyzed for intracellular IL-17 and surface MCAM levels as described in Materials and Methods above. As shown in FIG. 1C, although the majority of T cells producing IL-17 under these conditions were MCAM negative, MCAM protein was enriched on IL-17-producing cells. Only 2.3% of MCAM negative cells (2.18%/(2.18%+92.62%)) stained positive for IL-17; while 11.9% of MCAM expressing cells (0.62%/(0.62%+4.58%)) were IL-17 positive. Given these data, MCAM is enriched in IL-17-producing human CD4+ T cells.

Furthermore, when CD4+/CD45RO+ memory T cells were separated into purified populations of MCAM positive and MCAM negative cells and stimulated in vitro with anti-CD3 and anti-CD28, the MCAM positive population produced nearly ten times as much IL-17 (data not shown). The majority of the potential IL-17 production was found to be from the small population of T cells expressing MCAM. In one donor, only the MCAM positive population produced detectable levels of IL-17. Thus, the majority of the potential IL-17 production is from the small population of T cells expressing MCAM.

### Example 2. MCAM Expressing T Cells are Effector Memory T Cells Having a Unique Integrin Expression Profile

The CD45RO+ memory population of human CD4 T cells can be segregated into (1) effector memory cells with tissue tropism, and (2) central memory cells with lymphoid tissue homing based upon expression of CCR7. See, e.g., Sallusto et al., Nature 401: 708-712 (1999).

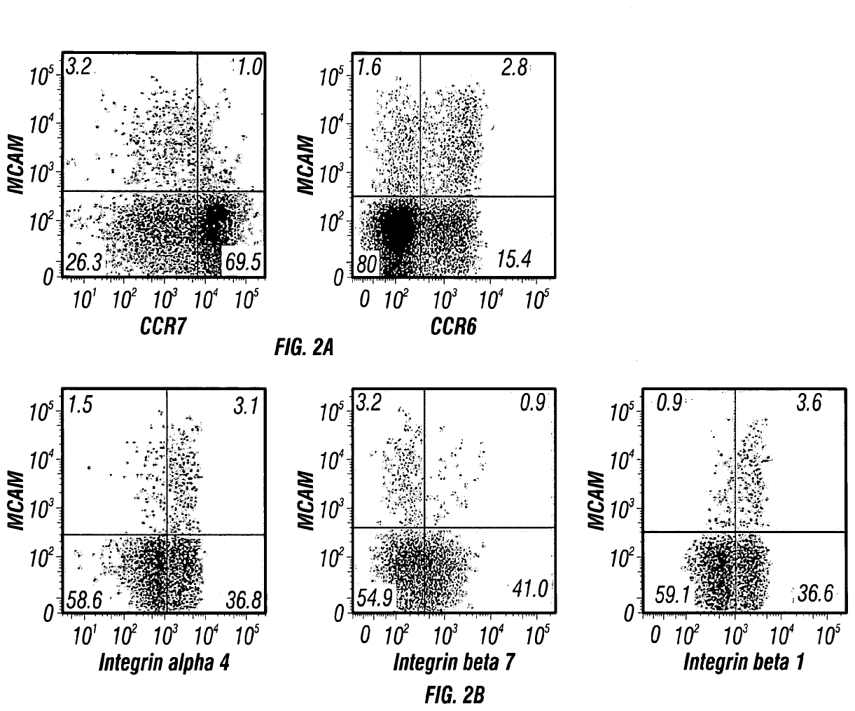

To determine which subpopulation includes the MCAM expressing T cells, MCAM expression in T cells was further characterized by staining peripheral human T cells with various markers (CCR6, CCR7, integrin subunits alpha 4, beta 1, and beta 7) as described in Materials and Methods above. MCAM expressing CD4+ T cells were largely CCR7 negative, indicating that most are effector memory T cells, and would be more likely to home to tissues (FIG. 2A). The TH17 enrichment protocol suggested that MCAM expressing T cells obtained would be disproportionately CCR6+. As shown in FIG. 2A, about 64% of MCAM+ cells (2.8%/(2.8%+1.6%)) express CCR6, while only 16.1% of MCAM negative cells (15.4%/(15.4%+80%)) express CCR6 (FIG. 2A). These data suggest that MCAM positive cells would be largely tropic for areas where the ligand for CCR6, CCL20, is high. See, e.g., Liao et al., J. Immunol. 162: 186-194 (1999).

The integrin expression pattern of MCAM expressing T cells was further characterized. The majority of MCAM expressing T cells are integrin α4 positive, but are largely integrin 137 negative and β1 positive (FIG. 2B), which is a phenotype associated with the T cells involved in the pathogenesis of EAE (experimental autoimmune encephalomyelitis). See, e.g., Bauer et al., Proc. Nat'l Acad. Sci. USA 106: 1920-1925 (2009).

### Example 3. MCAM Expressing T Cells are Expanded by IL1 and Produce the Majority of Both IL-17 and IL-22 Under TH17 Conditions

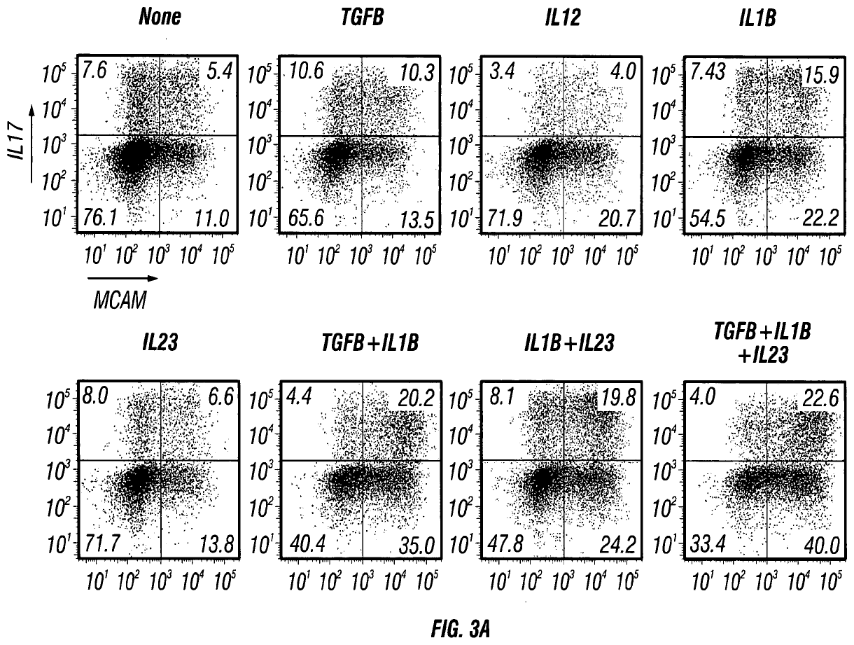

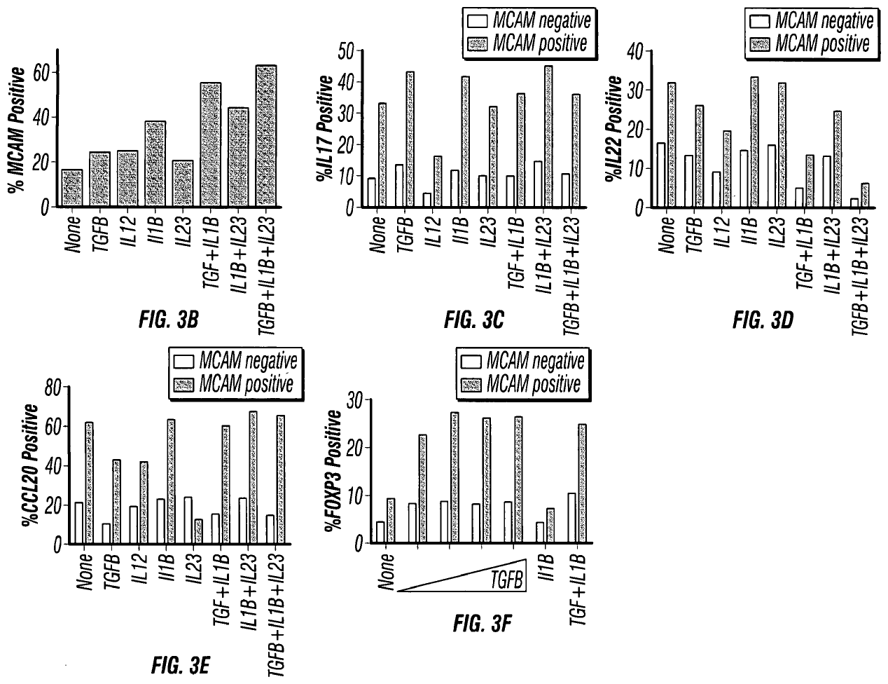

MCAM expressing CD4+ T cells, at only 3-5% of cells, is a small minority of the T cell population. It is of interest to determine the conditions under which this population expands and exerts TH17 effector function. For this, human CD4+/CD45RO+ T cells were purified as described in Materials and Methods above and stimulated in vitro with anti-CD3 and anti-CD28 in the presence of a number of cytokine conditions (TGFβ, IL-12, IL-1β, IL-23, and various combinations), and the percentage of MCAM expressing cells, as well as IL-17 expressing cells, was determined by flow cytometry (FIG. 3A). MCAM expression expanded upon stimulation with IL-1β alone (16.4% in the absence of IL-1β vs. 38.1% in the presence of IL-1β, FIG. 3B). Furthermore, while TGFβ alone did not expand the MCAM positive population greatly, it functioned synergistically with IL-1β, as the combination of both cytokines resulted in more than half of the memory T cell population becoming MCAM positive. Under the same conditions that expanded the population of MCAM expressing cells, the population of IL-17 producing cells was concomitantly increased, with considerable enrichment within the MCAM+ population under all cytokine conditions tested (FIG. 3C). In fact, in the presence of TGFβ and IL-1β, more than 80% of the IL-17 producing cells (20.2%/(20.2%+4.4%)) were MCAM positive.

Additional to IL-17, the known TH17 associated cytokine IL-22 (Liang et al., J. Exp. Med. 203: 2271-2279 (2006)) was also elevated in MCAM expressing T cells. IL-22 receptor is largely expressed on non-immune cells such as epithelial cells and functions in anti-microbial responses as well as tissue remodeling. See, e.g., Dumoutier et al., J. Immunol. 167: 3545-3549 (2001); see also Zenewicz et al., Int. Immunol. 23: 159-163 (2011). Although IL-22 has been shown to be involved in blood brain barrier function, it is not absolutely required for induction or progression of EAE. See, e.g., Kreymborg et al., J. Immunol. 179: 8098-8104 (2007); see also Kebir et al., Nat. Med. 13: 1173-1175 (2007). In a similar fashion to IL-17, a significantly higher percentage of MCAM+ cells expressed IL-22 (FIG. 3D).

TH17 cells have also been reported to express CCL20. See, e.g., Hirota et al., J. Exp. Med. 204: 2803-2812 (2007). Similar to IL-17 and IL-22, there was a considerably higher population of MCAM expressing T cells that were positive for CCL20 (FIG. 3E), suggesting a possible positive feedback loop in the migration of CCR6+ T cells.

While the above data are suggestive of a T cell population with a particularly pathogenic phenotype, it was unexpected to observe that MCAM expression was not mutually exclusive with intracellular FOXP3, and in fact, a slightly higher percentage of MCAM+ T cells were FOXP3 positive (FIG. 3F). In the presence of increasing doses of TGFβ, the percentage of MCAM+ cells that were FOXP3+ increased, while the percentage of FOXP3 expressing cells in the MCAM− population remained largely unchanged. These results suggest that MCAM expressing cells have the potential to function in an immunoregulatory role in the presence of TGFβ.

### Example 4. MCAM Binds to the ECM at Known Sites of T Cell Infiltration of the CNS, and the MCAM Ligand is Laminin 411

The function of MCAM has been elucidated in tumor models, showing that MCAM expression confers an adhesive, infiltrative, and ultimately metastatic phenotype to tumor cells. See, e.g., Xie et al., Cancer Res. 57: 2295-2303 (1997). However, the ligand that MCAM binds remains to be identified. Although the above data indicate that MCAM is enriched in TH17 cells, it is unknown whether MCAM is functionally involved in the T cell infiltration of the CNS. It was thus of great interest to determine (1) where MCAM binds, i.e., the identity of the MCAM ligand, (2) whether MCAM is critical to initial infiltration of TH17 cells into the uninflamed brain, and (3) whether the expression of the MCAM ligand is required at the established points of entry to the CNS.

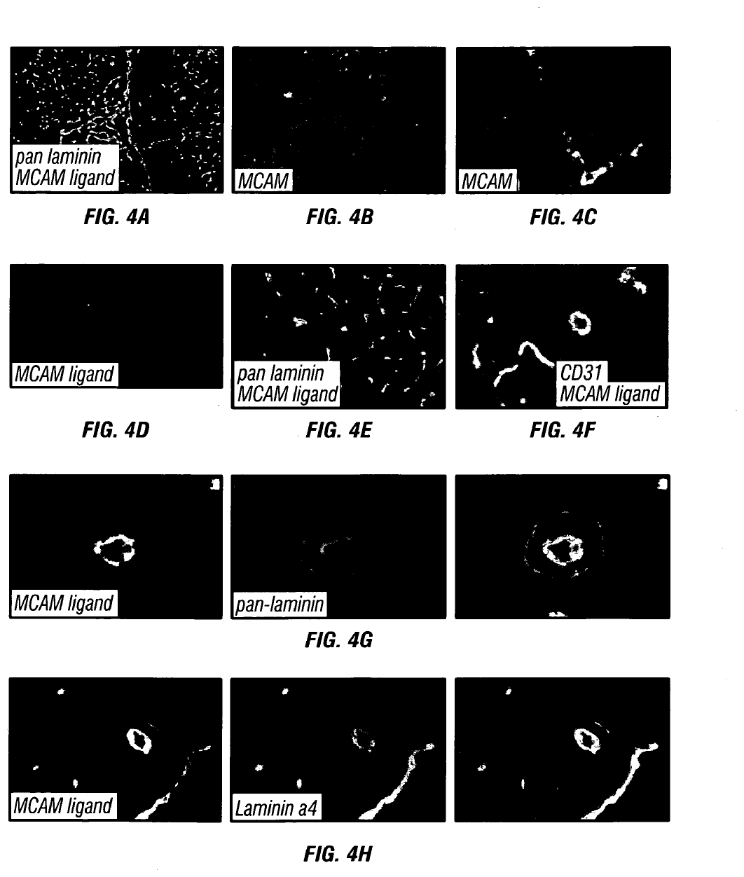

An MCAM-Fc fusion protein was generated (as described in Materials and Methods above) to detect MCAM binding on healthy mouse tissue, particularly those regions known to be involved in T cell infiltration. As the choroid plexus has been suggested as a route of entry for TH17 cells into the uninflamed brain, healthy choroid plexus tissue was stained with MCAM-Fc and anti-laminin. As shown in FIGS. 4A and 4B, the choroid plexus widely expresses the MCAM ligand, but is negative for MCAM. These results strongly suggest that (1) MCAM unlikely mediates adhesion to the choroid plexus tissue through a homotypic MCAM/MCAM interaction; and (2) there is an additional MCAM ligand with considerably more widespread expression than MCAM, whose expression was limited to vascular endothelium within healthy tissues (FIG. 4C). It was unexpected that MCAM-Fc bound nearly ubiquitously to healthy mouse spinal cord (FIG. 4D) in a pattern that was suggestive of an extracellular matrix (ECM) protein, and specifically laminin. MCAM-Fc and anti-laminin co-localized on healthy mouse spinal cord (FIG. 4E), suggesting that the ligand for MCAM might be a form of laminin. MCAM ligand was confirmed to be in the ECM, as it was exterior to the endothelial cell layer within the vasculature, as determined by CD31 co-staining (FIG. 4F).

While MCAM co-localized with laminin within healthy mouse tissues, the identity of the MCAM ligand was further confirmed by co-staining EAE tissues with laminin and MCAM-Fc. In regions of lymphocyte infiltration, it has been found that the basement membrane separates into two distinct membranes, the endothelial basement membrane and the parenchymal basement membrane with important distinctions in laminin isoform composition. See, e.g., Sixt et al., J Cell Biol. 153: 933-945 (2001). When MCAM-Fc was used to stain the MCAM ligand within these regions, it was found that MCAM-Fc stained only the endothelial basement membrane, while pan-laminin stained both the endothelia basement membrane and the parenchymal basement membrane (FIG. 4G). This same expression pattern has been noted for the laminin 411 (laminin 8 (α4β1γ1)). Co-localization of MCAM-Fc protein and laminin alpha 4 was observed by using a laminin alpha 4 specific antibody (FIG. 4H), suggesting that laminin 411 is a ligand for MCAM.

### Example 5. Anti-MCAM Antibodies Block Binding of MCAM to Laminin 411

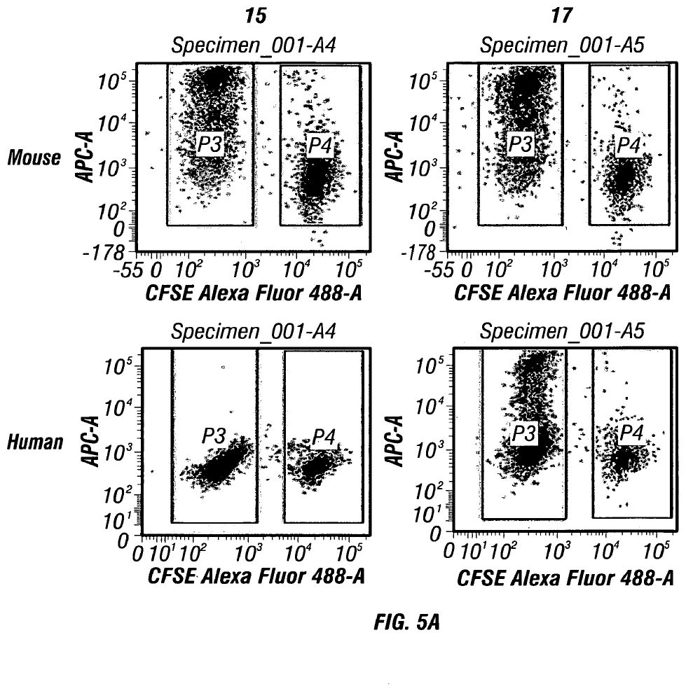

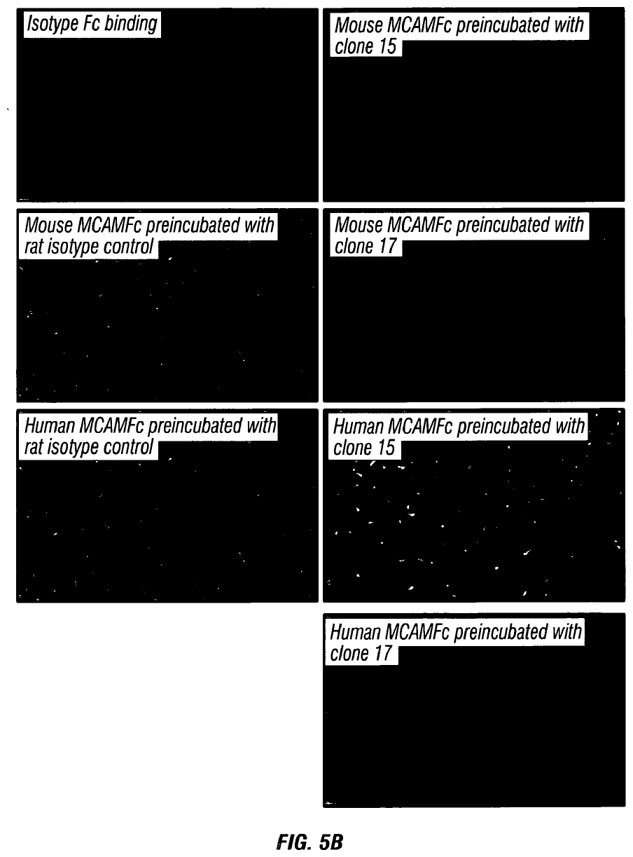

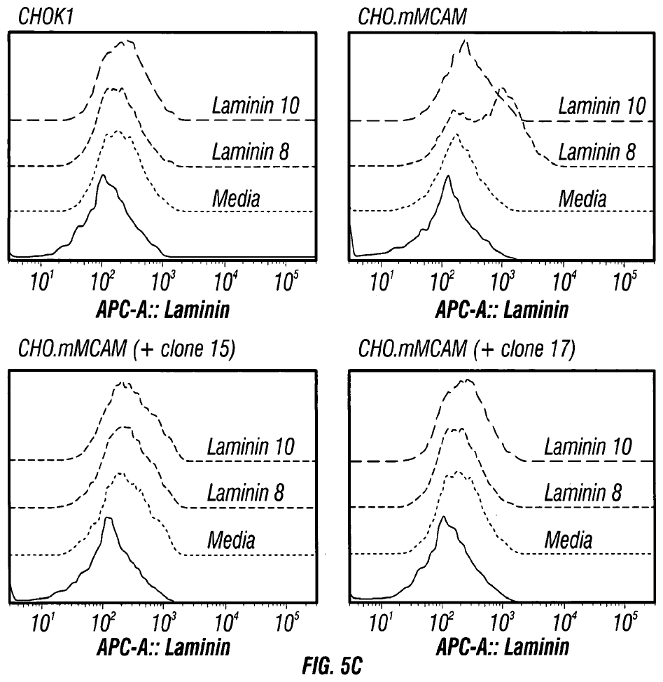

Monoclonal antibodies against mouse MCAM were generated as described in Materials and Methods above. The specific binding between the monoclonal antibody and MCAM was confirmed by assessing the monoclonal antibody's ability to bind to cells transfected with either mouse or human MCAM. For this, untransfected cells were labeled with carboxyfluorescein succinimidyl ester (CFSE) and mixed with unlabeled MCAM transfected cells. Untransfected cells (in blue) could therefore be differentiated. As shown in FIG. 5A, clones 15 and 17 showed specific binding to mouse MCAM (top, orange) while only clone 17 bound to human MCAM (bottom, orange).

Next, the monoclonal antibodies were used to test their ability to block the binding of MCAM to its ligand. Murine or human MCAM-Fc protein (5 μg/mL) was pre-incubated with isotype control antibody, clone 15, or clone 17 (10 μg/mL) for 30 minutes in PBS. The mixture was added to healthy spinal cord tissue sections and subsequently characterized by fluorescence microscopy as described in Materials and Methods above.

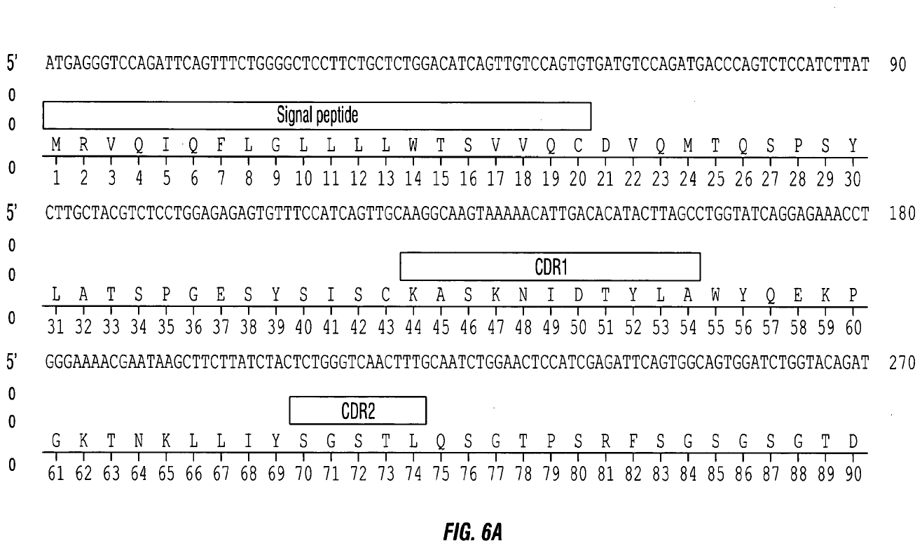

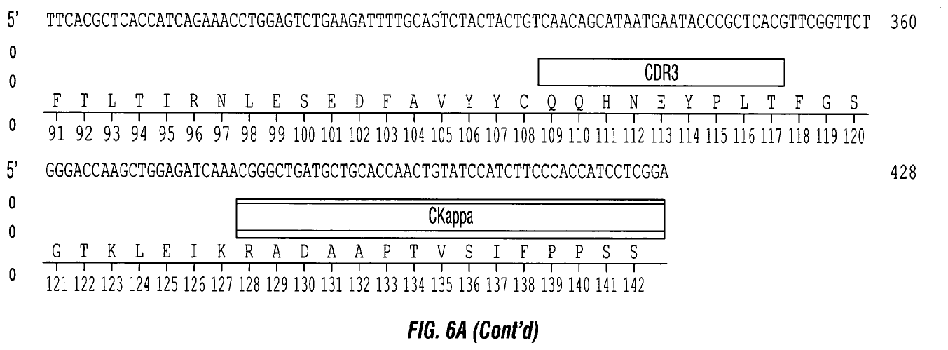

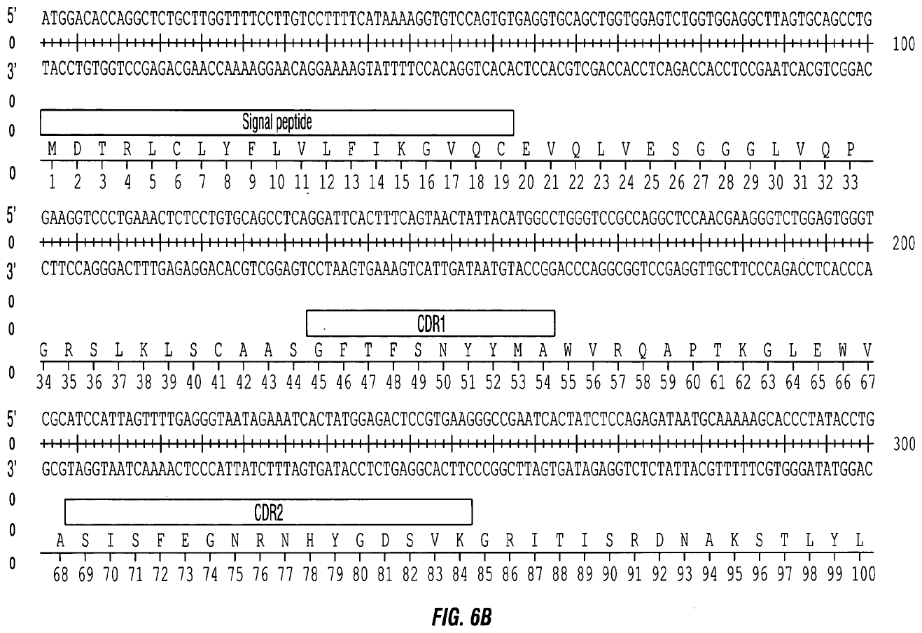

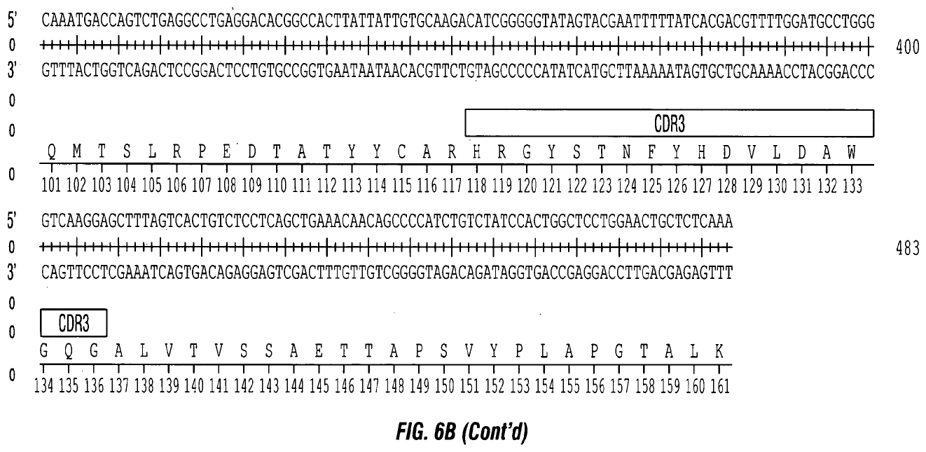

As shown in FIG. 5B, both clones 15 and 17 could block binding of the murine MCAM-Fc protein to the tissue, while only clone 17 could block human MCAM-Fc protein binding to the tissue. CDRs of clone 17 have been sequenced and are presented in FIGS. 6A (light chain) and 6B (heavy chain). Non-denaturing Western blot analysis using clone 17 on individual Fc domains of MCAM confirmed that clone 17 binds specifically to a domain comprising amino acid residues 19 to 129 of MCAM. This binding was confirmed by ForteBio analysis.

Furthermore, the MCAM monoclonal antibodies were shown to inhibit the interaction between MCAM and its ligand, laminin 411. Parental CHO cells (CHOK1) or CHO cells transfected with mouse MCAM gene were preincubated with Cho culture media (DMEM), recombinant laminin 411 (10 μg/ml), or recombinant laminin 511 (i.e., laminin 10 (α5β1γ1)) (10 μg/ml) at 37° C. for 45 minutes. Cells were washed, and specific binding of laminin 411, but not laminin 511, to MCAM was detected with a pan-laminin antibody by flow cytometry (FIG. 5C, top right panel). Preincubation of mouse MCAM transfected CHO cells with the anti-MCAM antibody (clone 15 or clone 17, each at 20 μg/ml), prior to laminin incubation, abolished the binding of MCAM to laminin 411 (FIG. 5C, bottom panels).

The above-presented data suggest that clone 17, which is capable of specifically blocking the binding of human MCAM to its ligand, might be useful to treat multiple sclerosis by inhibiting MCAM-mediated adhesion of TH17 cells to the vasculature and blocking the migration of TH17 cells into central nervous system.

### Example 6. MCAM is not Expressed on Circulating Mouse T Cells, but is Induced Following TH17 Polarization

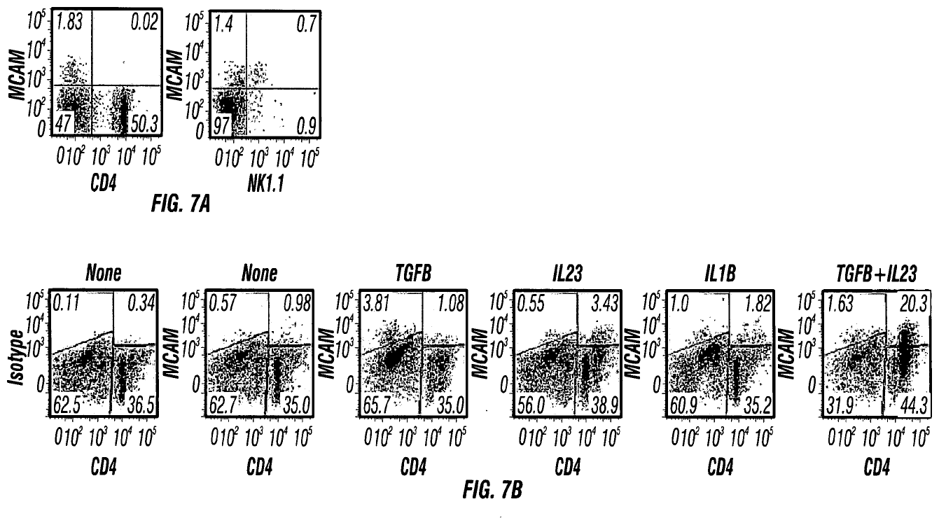

Using the antibodies above, peripheral mouse blood was stained to detect MCAM expressing T cells in mice as described in Materials and Methods above. As previously described, mouse T cells lack expression of MCAM, while expression is noted on a population of NK cells (FIG. 7A). The expression of MCAM solely on memory T cells in humans suggests that mice, if living in a clean environment with limited previous T cell activation, would have to be polarized in order to generate a population of MCAM expressing T cells. Considering the link between MCAM and TH17 cells in humans, experiments were conducted to determine whether it was possible to induce a population of MCAM expressing T cells in mice. Myelin proteolipid protein (PLP) specific T cells were generated by immunizing wild type mice with PLP in the presence of complete Freund's adjuvant (CFA) as described in Materials and Methods above. Splenocytes were restimulated in vitro with 5 μg/mL PLP in the presence of the indicated cytokines and analyzed five days later for MCAM expression (FIG. 7B). In the absence of exogenous cytokines, the restimulation did not induce statistically significant MCAM expression on CD4+ cells (as compared to isotype control). In the presence of IL-23, a small population of MCAM expressing CD4+ T cells was detectable. While TGFβ alone did not induce a sizable population of MCAM expressing T cells, the combination of TGFβ and IL-23 synergistically generated MCAM expression among CD4+ T cells. Both of these cytokines have an important role in the polarization and effector function of mouse TH17 cells. Notably, MCAM was expressed on a population of CD4 high T cells which have been described to exclusively contain the pathogenic T cells in EAE. See, e.g., Li et al., J. Neuroimmunol. 192: 57-67 (2007). Thus, unlike humans, mice do not possess a population of circulating CD4+ MCAM+ T cells, but polarization under TH17 conditions with TGFβ and IL-23 is sufficient to generate such a population. Mice remain a viable model to study the role of MCAM in the infiltration of CNS by pathogenic T cells.

### Example 7. MCAM Blockade by an Anti-MCAM Antibody Inhibits EAE Disease Progression

EAE is a disease that is generated laboratory animals to produce symptoms similar to those of multiple sclerosis (MS) in humans. EAE is generally produced by injecting animals with different proteins from the central nervous system of other animals, for example, extracts of myelin basic protein and whole spinal cord or brain tissue, or with T cells that specifically react to myelin. EAE is commonly used to follow the course the relapsing or progressive forms of MS. EAE has been served as a suitable animal model to both develop therapeutic agents for MS and study the specific disease processes of MS. See, e.g., Gold et al., Brain 129: 1953-1971 (2006); see also Steinman et al., Ann. Neurol. 60: 12-21 (2006).

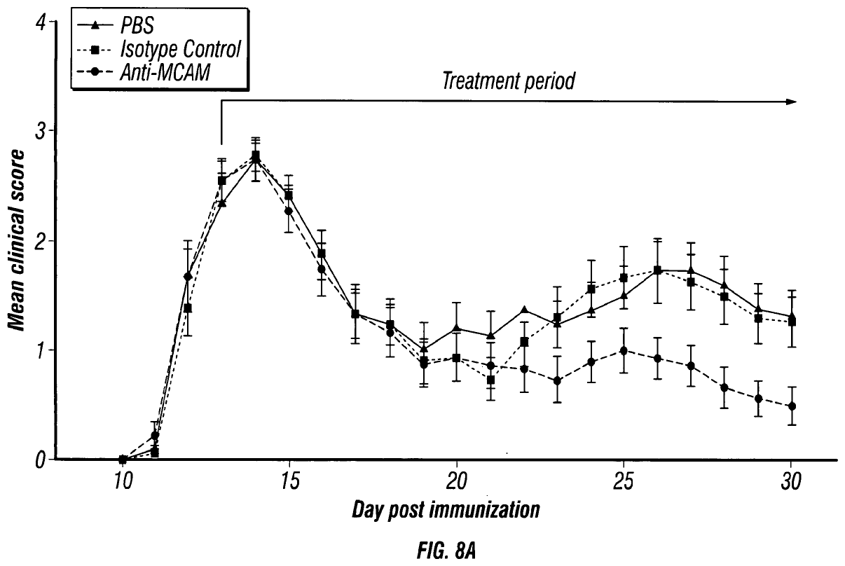

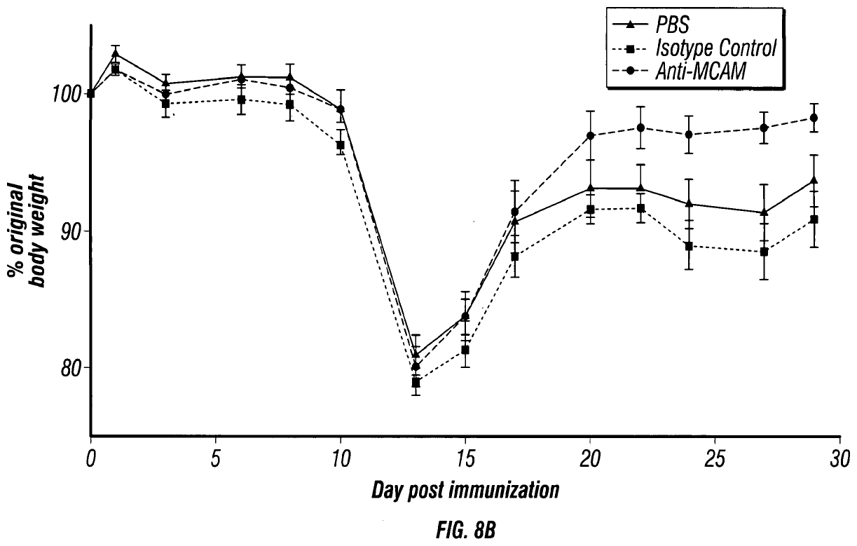

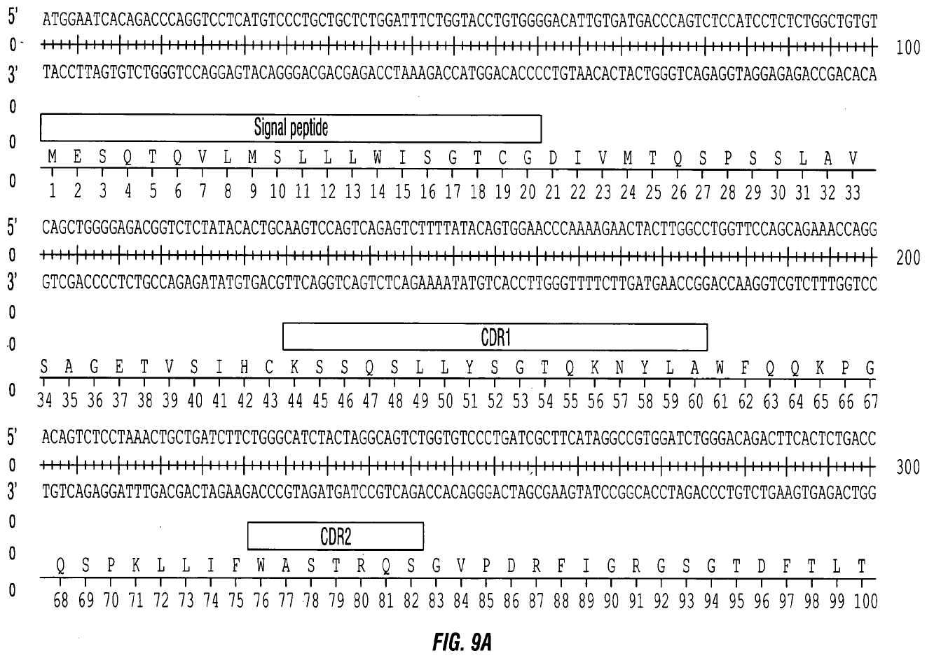

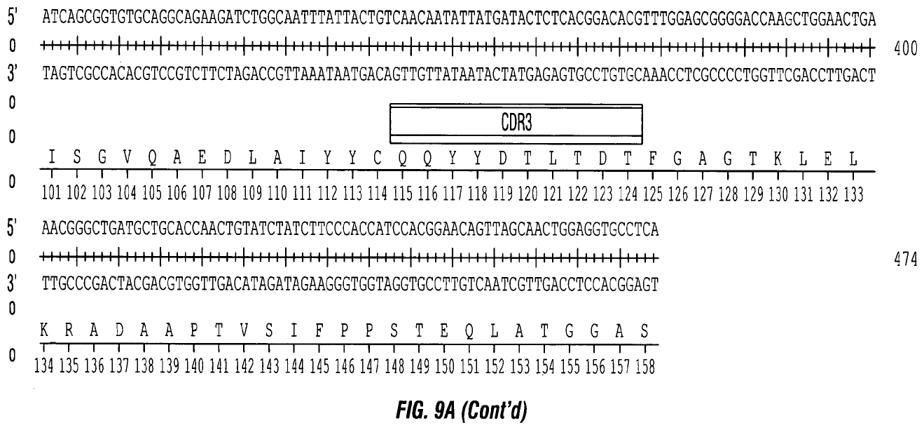

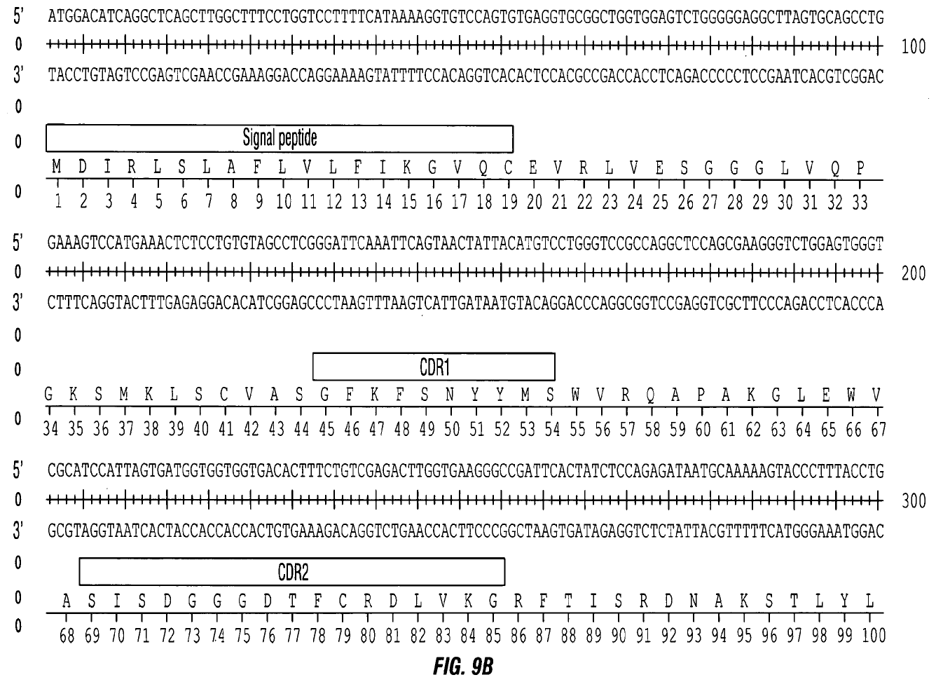

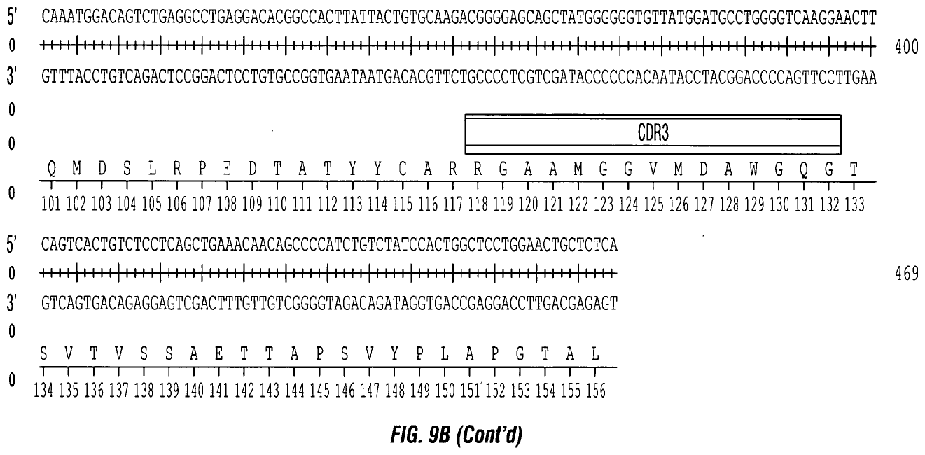

The effects of MCAM blockade on disease progression were further examined in a therapeutic model of EAE, wherein the TH17 polarization occurs in vivo (see Example 6). Mice were immunized with PLP 139-151 peptide as described in Materials and Methods above. Immunized mice were randomized into groups based on clinical scores and day of onset. On the second day following disease onset (EAE symptoms appeared between 12 and 14 days post immunization), mice were treated (N=15 per group) intraperitoneally with either anti-MCAM antibody (clone 15) or isotype control (Bioxcell) at 10 mg/kg body weight, and every day thereafter. Mice were monitored daily and scored for in a blinded manner (FIG. 8A), and body weights were obtained every 2-3 days (FIG. 8B). While MCAM blockade does not appear to affect the severity or duration of the ongoing acute phase of the disease, relapse was delayed and was significantly less severe in mice treated with anti-MCAM antibody (clone 15). These results are consistent with the idea that MCAM may not be essential for infiltration of immune cells during an existing inflammatory process, but may be involved in the subsequent recruitment of antigen experienced pioneer T cells to initiate new inflammatory sites.

### Example 8. Domain Binding Test for Murine Anti-MCAM Antibodies

The following protocol was used: ForteBio Domain Mapping Protocol. ForteBio anti-human IgG Fc biosensors were used to immobilize various mouse MCAMhFc domains including full length mouse MCAMhFc protein on to biosensor surface. These sensors were dipped into either clone 15 or 17 MCAM specific antibody for detection of binding to these domains or full length protein. After loading these samples into a black 96 well plate, the Octet Red was programmed as follows: 60 seconds for baseline #1; 180 seconds for loading various domains; 60 seconds for baseline #2; 180 seconds for association of antibody to domain; and 240 seconds for dissociation of antibody from domain.

Reagents and supplies used:

1. Mouse MCAMhFc final concentration @ 5 ug/ml

2. Rat antibody clone 15 or 17 @ 5 ug/ml

3. ForteBio anti-human IgG Fc Capture (AHC) biosensors for kinetics experiments, cat #18-5060

4. Block 96 well plate from Greiner Bio-one, cat #655209

5. ForteBio Octet Red machine

6. Fresh tissue culture medium, DMEM with 20% FCS, was used as buffer for dilution

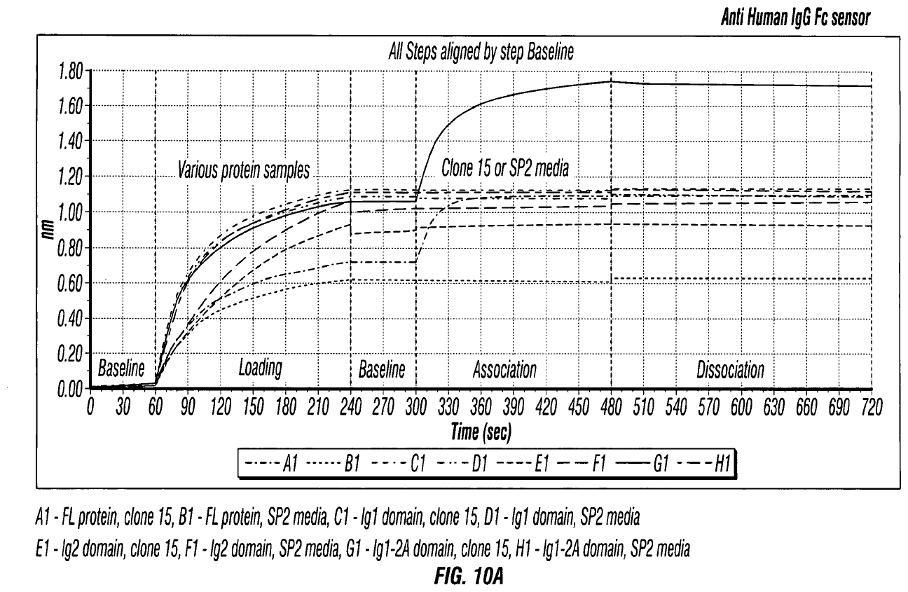

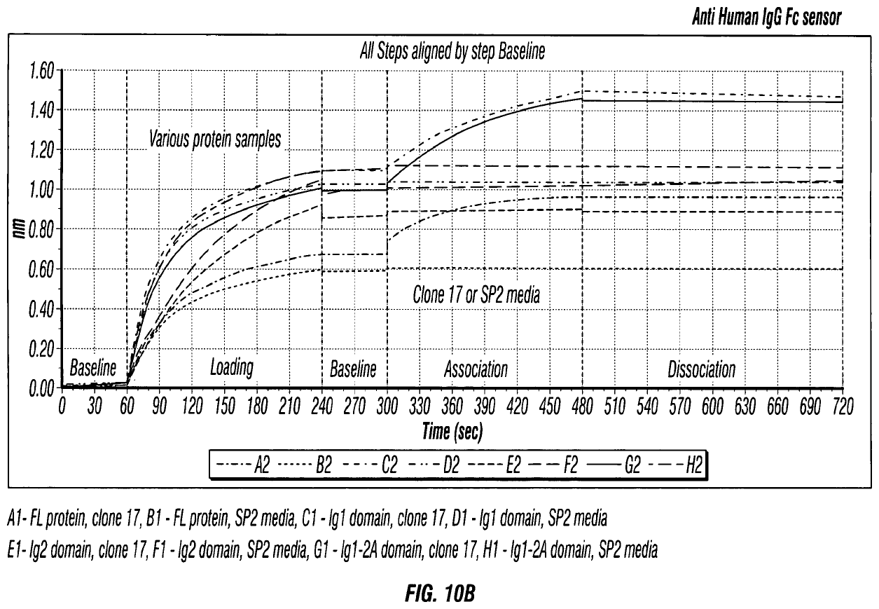

FIG. 10A demonstrates that clone 15 binds specifically to MCAM Fc domains 1 and 2, but not Fc domain 1 alone. FIG. 10B demonstrates that clone 17 binds specifically to either MCAM Fc domains 1 and 2, or Fc domain 1 alone. For FIG. 10A-B, clones 15 and 17 were tested against the following protein samples (all have human IgG Fc tag):

Murine MCAM; Human Fc full length protein; Murine MCAM domain 1 (Ig1); Murine MCAM domain 2 (Ig2); and Murine MCAM domain 1 and 2 (Ig1-2A).

### Example 9. MCAM Domains Bind Laminin A4 (α4) Chain

The binding affinity of the human laminin-α4 to human MCAM IgG1-2A was measured by Surface Plasmon Resonance on a Biacore T200 machine. Human Fc-specific F(ab′)₂ IgG (Jackson Laboratories) was immobilized on a CM5 chip using amine coupling. The four flow cells of the CM5 chips dextran surface are activated by a 7 min injection of freshly prepared 1:1 50 mM NHS: EDC at a flow rate of 5 μl/min. 70 μl IgG solution (pH 4.5) was injected for 3 min to a density of up to 3 000 RU. The coupling is then blocked by a 7 min injection of 1M ethanolamine to deactivate residual reactive sites. Recombinant human Fc-tagged MCAM IgG1-2A in degassed and filtered HBS-P buffer containing 12 mg/ml BSA and 12 mg/ml carboxy-methylated dextran sodium salt was captured by anti-Fc IgG to a capture level 1560 RU. Recombinant human Fc-tagged MCAM IgG1-2A was centrifuged at 14 000 rpm for 5 min at 4° C. before injection for 20 min at a flow rate of 5 μl/min over the anti-Fc IgG containing surface. Flow cell 1 was left free of IgG to serve as a control surface. One flow cell was used to capture recombinant human IgG1 Fc (R&D systems) to serve as a negative control. Recombinant human laminin-α4 (R&D systems) or recombinant human laminin 411 (Biolamina) or recombinant human laminin 511 (Biolamina) (negative control) was diluted in degassed and filtered HBS-P buffer containing 12 mg/ml BSA and 12 mg/ml carboxy-methylated dextran sodium salt to concentrations spanning 5-175 nM and injected (1 min association, 3 min dissociation) over the MCAM IgG1-2A surfaces and control surfaces at a flow rate of 10 μl/min. Buffer injections served as negative control. Data evaluation: Data from the buffer injections and the control surface were subtracted to remove artifacts. The data was fitted globally to a 1:1 interaction model using the Biaevaluation software or Scrubber.

The laminin 4A chain was found to bind specifically to the MCAM Fc domains 1 and 2, but not to Fc domain 1 alone (data not shown). The negative controls included: a lack of binding of laminin 511 to either domain, and a lack of binding of laminin 411 to hIgG1-Fc. Recombinant human laminin-α4 (R&D systems) binds to human Fc-tagged MCAM IgG1-2A (data not shown) at an affinity of 60 nM, but not to recombinant human IgG1 Fc (R&D systems) (data not shown). Recombinant human laminin 411 (Biolamina) binds to human Fc-tagged MCAM IgG1-2A at an affinity of 66 nM as measured by steady state kinetics (data not shown) but not to recombinant human IgG1 Fc (R&D systems) (data not shown). The negative control, recombinant human laminin 511 (Biolamina) does not bind to human Fc-tagged MCAM IgG1-2A (data not shown).

All literature and patent references cited above are herein incorporated by reference in their entirety.

1-38. (canceled)

39. A method of obtaining an antibody that inhibits MCAM binding to laminin-alpha-4, comprising:
preparing an antibody that binds to MCAM or laminin α4; and
assaying the antibody to determine it inhibits binding of MCAM to laminin α4.

40. The method of claim 40, wherein the antibody is monoclonal.

41. The method of claim 40, wherein the preparing is performed by immunizing a mouse or rat with an MCAM extracellular domain.

42. The method of claim 40, wherein the determining is performed by incubating cells expressing MCAM with laminin α4 in the presence of the monoclonal antibody.

43. The method of claim 40, wherein the determining is performed by incubating cells expressing laminin α4 with MCAM or a fragment thereof in the presence of the monoclonal antibody.

44. The method of claim 40, further comprising mapping the epitope of the monoclonal antibody.

45. The method of claim 40, wherein the monoclonal antibody is prepared by immunizing a non-human animal with an antigenic MCAM epitope.

46. The method of claim 40, wherein the monoclonal antibody is prepared by phage display.

47. The method of claim 40, further comprising preparing a humanized or chimeric form of the antibody.

48. The method of claim 40, further comprising incorporating the monoclonal antibody into a pharmaceutical composition.

49. The method of claim 40, further comprising determining the monoclonal antibody inhibits binding of a reference antibody to MCAM or laminin-alpha-4.

50. The method of claim 40, wherein the antibody inhibits the interaction of an MCAM domain comprising SEQ ID NO:22 and/or SEQ ID NO:23 with a laminin-alpha-4 chain.
